(19) United States
(12) Patent Application Publication
(10) Pub. No.: US 2025/0010022 A1
(43) Pub. Date: Jan. 9, 2025

(54) CATHETER ASSEMBLY

(71) Applicant: ConvaTec Limited, Flintshire (GB)

(72) Inventors: Oliver Pfleger, Spital (GB); Lukasz Natkaniec, Munchen (DE); Phillip Emrich, Munchen (DE)

(21) Appl. No.: 18/891,011

(22) Filed: Sep. 20, 2024

Related U.S. Application Data
(63) Continuation of application No. PCT/GB2023/050673, filed on Mar. 20, 2023.

(30) Foreign Application Priority Data
Mar. 21, 2022 (GB) GB2203905.1

(51) Int. Cl. A61M 25/00; A61M 39/22

(52) U.S. Cl. CPC A61M 25/0017; A61M 25/0009; A61M 25/002; A61M 39/22; A61M 2205/0216; A61M 2205/0255; A61M 2205/0261; A61M 2205/0266; A61M 2207/10; A61M 2209/06; A61M 2210/1089

(57) ABSTRACT

An intermittent catheter assembly that has a catheter having a proximal end for insertion into the body and a distal end, a fluid collection bag arranged to receive fluid from the distal end of the catheter, wherein the fluid collection bag has an outlet valve configured to control release of fluid out of the fluid collection bag in use and the outlet valve has a valve base attached to a panel of the fluid collection bag, the valve base having a base opening for receiving a liquid output from the fluid collection bag and an outlet element configured to be laterally rotatable relative to the valve base from a closed configuration to an open configuration.

### CROSS REFERENCE TO RELATED DISCLOSURE

The present disclosure is a Continuation of International Application No. PCT/GB2023/050673 filed Mar. 20, 2023 and claims the benefit of GB 2203905.1 filed Mar. 21, 2022, the contents of which are incorporated herein by reference in entirety.

### TECHNICAL FIELD OF THE INVENTION

The present invention relates to catheter assemblies. In particular the invention concerns packaged urinary catheter assemblies, and most particularly, but not exclusively packaged intermittent male urinary catheter assemblies, especially “closed” catheter assemblies.

### BACKGROUND TO THE INVENTION

Many people suffer from urinary problems that can make it difficult to pass urine. Intermittent catheter assemblies can provide a convenient and portable solution to this problem as a user is able to self-catheterise to relieve themselves as required. This reduces the effect of their urinary condition on their life and allows them to enjoy a relatively normal lifestyle.

Many existing catheter assemblies require the catheter to be strapped to a user's body, typically the user's leg to support the fluid collection bag when in use. This is conventional when the catheter is a permanent catheter, such as a Foley catheter. For intermittent catheters, that is, catheters which are used intermittently throughout a day for short term catheter-based management of the bladder, it is not practical or comfortable for the user to have a catheter strapped to their body for the entire day when the catheter is intended to be inserted into the user only long enough to drain the bladder, then removed. As such, when a user intends to use an intermittent catheter, they must either hold the fluid collection bag during use or find a suitable place to rest the bag. In each instance, this is cumbersome and creates problems for the user, in particular for users with limited dexterity. It must be appreciated that holding a fluid collection bag by the bag itself during urination is increasingly difficult when the bag is being filled with liquid. This is because the user may grip the bag too tightly, which can disrupt the filling of the bag, cause the distal end of the catheter to detach from the bag, or cause the bag to overflow with liquid. In each case, what should be a private and discrete moment for the user, can become problematic and embarrassing.

In some existing catheter assemblies, the fluid collection bag is provided with an outlet valve for releasing the contents of the bag after use. This is particularly common in permanent catheter assemblies, such as a Foley catheter. Permanent catheters are attached, for example by a strap, to the body of a user, typically the user's leg. As such, movement of the fluid collection bag is prevented when the user operates the outlet valve. Moreover, permanent catheters comprise a ‘north-south’ outlet valve, meaning that the valve must be actuated in an upward and downward motion to open and close the valve. Thus, the valve is arranged parallel to the user's leg, so the user does not accidentally open the valve when crossing their legs, as lateral movement will not open the valve. Moreover, the ‘north-south’ outlet valve on a catheter of this type is typically operable by a user who is fully dextrous (having the Foley catheter as a temporary measure) or by a healthcare professional on the user's behalf, where the user is bedridden. However, if the user has limited dexterity or the catheter assembly was not attached to the user's body, for example where the catheter is an intermittent catheter, then ‘north-south’ outlet valves would be cumbersome to operate as they require the fluid collection bag to be held whilst operating the valve. This can be problematic for the user making the outlet valves difficult to operate.

To reduce the risk of damage to the urinary tract and improve comfort while using the catheter, it is also important they are activated before use for example through wetting of the catheter by a wetting fluid. However, once wetted and/or used a catheter can be slippery and difficult to handle. This can make handling and re-packaging of the catheter after use difficult and unhygienic, as the catheter may inadvertently contact the user's hands, clothing or personal items. Existing intermittent catheters disclose continuous handling sleeves that cover the entire length of the catheter. While these provide some assistance to handling, the sleeve can make the catheter more difficult to wet as wetting fluid may not be able to easily reach all parts of the catheter.

When using a catheter, it is important that the catheter remains clean but when packaged this can be challenging as the catheter must be removed from the packaging. This can lead to the user touching a part of the catheter that is intended to be introduced into the body, such as the tube, by accident while they attempt to remove the catheter from the packaging which can lead to a risk of infection during use. It is therefore important to provide packaging which is convenient and easy for the user to open and which presents the catheter to the user such that it can be removed from the packaging for use without accidentally touching a part of the catheter that should remain clean directly.

To provide discrete and portable catheter assemblies, packaging may be provided to contain the catheter and any other equipment such as wetting reservoirs or sachets and fluid collection bags before and/or after use. However, these items may become wet which makes handling and re-packaging of them difficult. In addition, where liquid is contained within a sachet, reservoir or bag within the packaging, any release (intentional or otherwise) of this may cause liquid to accidentally leak out onto parts of the catheter assembly that the user has to touch—such as a handling element, or onto the packaging causing problems for the user.

It is an object of embodiments of the present invention to at least partially overcome or alleviate the above problems and/or to provide an improved catheter assembly.

### SUMMARY OF THE INVENTION

According to a broad aspect of the present invention, there is provided a fluid collection bag for a catheter assembly, wherein the fluid collection bag comprises a hand strap.

‘Hand strap’ as used herein means a strap sized to be easily gripped by hand.

The fluid collection bag may be provided in a packaged catheter assembly, the packaged catheter assembly may comprise a pouch, a catheter, and the fluid collection bag. The packaged catheter assembly may comprise a fluid reservoir. The fluid collection bag may be configured to receive fluid from the catheter. The catheter, fluid reservoir and fluid collection bag may be arranged within the pouch. The fluid collection bag may comprise a front panel and a rear panel. A bond between the two panels may define a (internal) cavity.

The fluid collection bag of the broad aspect of the invention may comprise any of the features, including any optional features, as described in relation to the first aspect, or any other aspect, of the invention below.

According to a first aspect of the present invention, there is provided an intermittent catheter assembly comprising: a catheter having a proximal end for insertion into the body and a distal end; and a fluid collection bag arranged to receive fluid from the distal end of the catheter, wherein the fluid collection bag comprises a hand strap.

Advantageously, if the user is to hold the fluid collection bag when in use, the hand strap provides for a convenient and easy way of holding the fluid collection bag, where the hand strap can be temporarily worn around the user's wrist or be easily held. As such, the provision of a hand strap overcomes the problems of prior art intermittent catheters and ensures that the catheter is easy and convenient to use, in addition to being discrete and hygienic.

Further, the hand strap allows the fluid collection bag to be easily manipulated by the user during use. This means that the bag can be easily handled and manoeuvred by the user as is necessary. It also means that the user is no longer relying on their surroundings to use the catheter. For example, catheters described in the prior art are difficult to hold during use and so may need to be placed on the floor or leaning against an object to support the fluid collection bag during use. As stated above, this is associated with its own disadvantages. The present invention provides an assembly which can be easily and reliably held by the user.

Moreover, the provision of a hand strap means that the fluid collection bag can be hung from any suitable article, such as a wheelchair handle or bed post, when the catheter assembly is in use, i.e., during urination. Beneficially for the user, this means that the user does not need to transfer to a toilet cubicle or place the bag on the floor, which is plainly unhygienic, in order to urinate.

The fluid collection bag may receive fluid via an inlet arranged at an edge of the fluid collection bag. The hand strap may be arranged on the same edge as the inlet.

Advantageously, this means that when the fluid collection bag is held via the hand strap, for example by a user or by an article such as a wheelchair handle, the fluid entering the fluid collection bag flows away from the inlet, preventing back flow of the fluid toward the inlet and then the catheter, which can prevent the bag from being filled. Further advantageously, this provides for easier manipulation of the bag relative to the inlet. In use, it may be that the user will have to adjust the catheter and this will likely affect the position of the catheter relative to the fluid collection bag. As such, it is beneficial that the present invention provides for a fluid collection bag which can be easily manoeuvred and adjusted in use.

The fluid collection bag may comprise a front panel and a rear panel, and a bond between the two panels defining an internal cavity. The bond may be a peripheral bond. The peripheral bond may define a base, two lateral edges and an upper edge of the fluid collection bag. The two lateral edges may comprise a right lateral edge and a left lateral edge. The right lateral edge and left lateral edge may be defined as the right and left sides of the bag when viewing the bag with the rear panel behind the front panel, the base at the bottom of the bag and the upper edge at the top of the bag. The bag may have a width between the left lateral edge and right lateral edge of at least 8, 10, 12, 15 or 20 cm. The bag may have a width no more than 25, 20, 15, 10 or 8 cm. Preferably, the width is between 8 and 20 cm, more preferably between 10 and 15 cm, and most preferably about 12 cm. The base may define a bottom of the bag. The upper edge may define a top of the bag. The height of the bag from the base to the upper edge may be at least 20, 25, 30, 35 or 40 cm. The height of the bag may be no more than 45, 40, 35, 30, 25 or 20 cm. The height may be between 20 and 40 cm, 25 and 35 cm, or most preferably between 28 and 32 cm, for example 30 cm. The bag may be configured to hold at least 500, 700, or 1000 ml of fluid, and may hold no more than 1000 ml, for example, it may hold at least 700 ml of fluid.

The inlet may be arranged at the upper edge, left lateral edge, right lateral edge, or base of the fluid collection bag. Preferably, the inlet is arranged at the upper edge of the fluid collection bag. The hand strap may be arranged at the upper edge, left lateral edge, right lateral edge, or base of the fluid collection bag. Preferably, the hand strap is arranged at the upper edge of the fluid collection bag.

The hand strap may be arranged on a corner of the fluid collection bag where the upper edge and left lateral edge meet, a corner where the upper edge and right lateral edge meet, a corner where the left lateral edge and base meet, or a corner where the right lateral edge and base meet.

The fluid collection bag may comprise an outlet provided to allow fluid to pass out of the fluid collection bag after use. The hand strap may be arranged at an opposite end of the fluid collection bag to the outlet. Advantageously, this means that, in use, the bag is arranged such that the fluid entering the bag flows toward the outlet, preferably at or toward the base of the bag, irrespective of the position of the inlet. This facilitates draining of the bag during and/or after use. Moreover, when the user wishes to drain the bag, the bag can be held via the hand strap and the fluid discharged from the bag via the outlet, therefore, the user is not required to upend the bag to pour away the contents, which can be cumbersome.

The outlet may be arranged at the upper edge, left lateral edge, right lateral edge, or base of the fluid collection bag. The outlet may be arranged on the front panel or rear panel of the fluid collection bag, toward the upper edge, left lateral edge, right lateral edge or base. Preferably, the outlet is arranged on the front panel toward the base of the fluid collection bag.

The outlet may be arranged off-centre when the fluid collection bag is viewed with the rear panel behind the front panel. The outlet may be arranged off-centre, toward the left lateral edge or toward the right lateral edge.

The outlet may be arranged off-centre toward the side of the fluid collection bag on which the hand strap is arranged. For example, the hand strap may be arranged on the corner where the upper edge and right lateral edge meet, and the outlet may be arranged proximal to the base, off-centre toward the right lateral edge.

The hand strap may be joined to the fluid collection bag by any one or more of: a weld; mechanical seal; heat seal; pressure seal; adhesive; solvent bond; ultraviolet bond; ultrasonic weld; laser weld; impulse weld; or friction weld. In a preferred embodiment, the hand strap is joined to the bag by a weld. Advantageously, the hand strap may be joined to the bag easily and added to the fluid collection bag construction as part of conventional techniques for the manufacture of fluid collection bags.

The length of the hand strap may be between about 4 and about 30 cm, between about 5 and about 28 cm, between about 6 and about 26 cm, between about 7 and about 24 cm, between about 8 and about 22 cm, between about 9 and about 20 cm, between about 10 and about 19 cm, between about 11 and about 18 cm, between about 12 and about 17 cm, between about 13 and about 16 cm, between about 14 and about 16 cm, or about 15 cm. Advantageously, this provides a hand strap which can be arranged around many articles which a user may wish to hang the hand strap upon, such as a wheelchair handle or bed frame.

The length of the hand strap may be at least about 4 cm, about 5 cm, about 6 cm, about 7 cm, about 8 cm, about 9 cm, about 10 cm, about 11 cm, about 12 cm, about 13 cm, about 14 cm, about 15 cm, about 16 cm, about 17 cm, about 18 cm, about 19 cm, about 20 cm, about 22 cm, about 24 cm, about 26 cm, about 28 cm or at least about 30 cm.

The length of the hand strap may be no more than about 4 cm, about 5 cm, about 6 cm, about 7 cm, about 8 cm, about 9 cm, about 10 cm, about 11 cm, about 12 cm, about 13 cm, about 14 cm, about 15 cm, about 16 cm, about 17 cm, about 18 cm, about 19 cm, about 20 cm, about 22 cm, about 24 cm, about 26 cm, about 28 cm or no more than about 30 cm.

The hand strap may have a length equal to between about 20 and 100%, between about 30 and about 90%, between about 35 and about 80%, between about 35 and about 70%, between about 40 and about 60%, between about 45 and about 55%, or about 50% of the length of the fluid collection bag.

The hand strap may have a length equal to at least about 20%, about 30%, about 40%, about 50%, about 60%, about 70%, about 80%, about 90%, or at least about 100% of the length of the fluid collection bag.

The hand strap may have a length equal to no more than about 20%, about 30%, about 40%, about 50%, about 60%, about 70%, about 80%, about 90%, or no more than about 100% of the length of the fluid collection bag.

The size of the hand strap may be fixed, i.e., the size of the hand strap may not be adjustable. This means that the length of the hand strap cannot be decreased or increased. This may provide a more secure hand strap because means for shortening or lengthening the strap can create areas of weakness in the hand strap, which can cause the strap to break after repeated use or increased tension.

Alternatively, the size of the hand strap may be adjustable such that the length of the hand strap can be decreased or increased.

The hand strap may have a width of between about 0.5 and about 4 cm, between about 0.6 and about 3.6 cm, between about 0.6 and about 3.2 cm, between about 0.7 and about 2.8 cm, between about 0.75 and about 2.4 cm, between about 0.8 and about 2 cm, between about 0.85 and about 1.6 cm, between about 0.9 and about 1.2 cm, between about 0.95 and about 1.1 cm, or about 1 cm.

The hand strap may have a width of at least about 0.5 cm, about 0.6 cm, about 0.7 cm, about 0.8 cm, about 0.9 cm, about 1 cm, about 1.5 cm, about 2 cm, about 2.5 cm, about 3 cm, about 3.5 cm, about 4 cm, about 4.5 cm, or at least about 5 cm.

The hand strap may have a width of no more than about 0.5 cm, about 0.6 cm, about 0.7 cm, about 0.8 cm, about 0.9 cm, about 1 cm, about 1.5 cm, about 2 cm, about 2.5 cm, about 3 cm, about 3.5 cm, about 4 cm, about 4.5 cm, or no more than about 5 cm.

The width of the hand strap may be constant along its entire length.

The hand strap may be a loop of material or a strip of material. The hand strap may be a single, continuous loop of material.

Where the hand strap is a strip of material it may be attached at only one end to the fluid collection bag. Where the hand strap is a loop of material, it may be attached at two ends to the fluid collection bag. Where the hand strap is a loop of material, it may be joined to the fluid collection bag at two positions on the fluid collection bag. Each of the two positions may be substantially the same.

The hand strap may be joined to the fluid collection bag at a first position and at a second position. Preferably, the first position and the second position are substantially the same. The first position and the second position may each be on the upper edge, left lateral edge, base, right lateral edge, front panel, or rear panel of the fluid collection bag. The first position may be on a different edge or panel to the second position. For example, the first position may be on the front panel and the second position may be on the rear panel. As a further example, the first position may be on the upper edge and the second position may be on the rear panel.

The hand strap may be joined to the fluid collection bag via an aperture on the surface of the bag, and the hand strap is threaded through the aperture. The aperture may be provided on an edge of the fluid collection bag, preferably on the upper edge of the bag.

The hand strap may be formed of polyethylene terephthalate (PET), polypropylene or low-density polyethylene (LDPE).

The catheter may have a length of at least about 10 cm, about 15 cm, about 20 cm, about 25 cm, about 30 cm, about 35 cm, about 40 cm, about 45 cm, or about 50 cm.

The catheter may have a length of no more than about 70, 60, 50, 45, 40 or 35 cm.

The catheter may have a width of no more than about 10 cm, about 15 cm, about 20 cm, about 25 cm, about 30 cm, about 35 cm, about 40 cm, about 45 cm, about 50 cm, about 55 cm, about 60 cm, about 65 cm, about 70 cm, about 80 cm, about 90 cm, or no more than about 100 cm.

The intermittent catheter may be formed of a material of the group comprising: polyvinyl chloride, polytetrafluoroethylene, polyolefins, latex, silicones, synthetic rubbers, polyurethanes, polyesters, polyacrylates, polyamides, thermoplastic elastomeric materials, styrene block copolymers, polyether block amide, thermoplastic vulcanizates, thermoplastic copolyesters, thermoplastic polyamides, and water disintegrable or enzymatically hydrolysable material, or combinations, blends or co-polymers of any of the above materials.

The intermittent catheter may be formed of a material of the group comprising: polyolefins, polyesters, polyacrylates, polyamides, thermoplastic elastomeric material, polyether block amide, thermoplastic vulcanizates, thermoplastic copolyesters, thermoplastic polyamides, fluororubber, and water disintegrable or enzymatically hydrolysable material or combinations, blends or co-polymers of any of the above materials.

The water disintegrable or enzymatically hydrolysable material may comprise a material of the group comprising: polyvinyl alcohol, extrudable polyvinyl alcohol, polyacrylic acids, polylactic acid, polyesters, polyglycolide, polyglycolic acid, poly lactic-co-glycolic acid, polylactide, amines, polyacrylamides, poly(N-(2-Hydroxypropyl) methacrylamide), starch, modified starches or derivatives, amylopectin, pectin, xanthan, scleroglucan, dextrin, chitosans, chitins, agar, alginate, carrageenans, laminarin, saccharides, polysaccharides, sucrose, polyethylene oxide, polypropylene oxide, acrylics, polyacrylic acid blends, poly(methacrylic acid), polystyrene sulfonate, polyethylene sulfonate, lignin sulfonate, polymethacrylamides, copolymers of aminoalkyl-acrylamides and methacrylamides, melamine-formaldehyde copolymers, vinyl alcohol copolymers, cellulose ethers, poly-ethers, polyethylene oxide, blends of polyethylene-polypropylene glycol, carboxymethyl cellulose, guar gum, locust bean gum, hydroxypropyl cellulose, vinylpyrrolidone polymers and copolymers, polyvinyl pyrrolidone-ethylene-vinyl acetate, polyvinyl pyrrolidone-carboxymethyl cellulose, carboxymethyl cellulose shellac, copolymers of vinylpyrrolidone with vinyl acetate, hydroxyethyl cellulose, gelatin, poly-caprolactone, poly(p-dioxanone), or combinations, blends or co-polymers of any of the above materials.

Preferably, the intermittent catheter is formed of a polyolefin material, in particular polyethylene and/or polypropylene.

Preferably, the intermittent catheter is formed of a thermoplastic elastomeric material.

At least a portion of the fluid collection bag may be formed from an opaque material or be coated to present the appearance of being opaque. At least 10%, 20%, 30%, 40%, 50%, 60%, 70% or at least 80% of the fluid collection bag may be formed from an opaque material or be coated to present the appearance of being opaque. No more than 10%, 20%, 30%, 40%, 50%, 60%, 70% no more than 80% of the fluid collection bag may be formed from an opaque material or be coated to present the appearance of being opaque. The fluid collection bag may be opaque at the inlet and/or outlet. Advantageously, this makes the contents of the pouch during and after use at least partially hidden and, therefore, more discrete for the user.

The inlet may be located at the end of a spout. The spout may extend from the upper edge, left lateral edge, base or right lateral edge of the fluid collection bag, but preferably from the upper edge. The diameter of the spout may remain constant for its entire length. The spout may comprise an end, distal to the fluid collection bag, which comprises the inlet. The spout may have a length, i.e., the distance from where the spout joins an edge of the fluid collection bag to the inlet, of between 2 and 14 cm, between 4 and 12 cm, between 6 and 10 cm, between 7 and 9 cm, or about 8 cm.

Advantageously, the spout makes it easier for the user to locate the inlet and provides a more secure connection between the catheter and the fluid collection bag.

The fluid collection bag may comprise an outlet valve configured to control release of fluid out of the fluid collection bag in use. The outlet valve may comprise: a valve base attached to a panel of the fluid collection bag, the valve base comprising a base opening for receiving a liquid output from the fluid collection bag; and an outlet element configured to be laterally rotatable relative to the valve base from a closed configuration to an open configuration.

The outlet element may comprise a handle operable to rotate the outlet element between the closed configuration and the open configuration. The handle may be planar.

The plane of handle may be aligned with, i.e., parallel with, the plane of the panel of the fluid collection bag to which the valve base is attached, when in the closed configuration.

According to a second aspect of the invention, there is provided a packaged catheter assembly comprising the intermittent catheter assembly of the first aspect, wherein the intermittent catheter assembly is arranged within a pouch.

The pouch may be sealed by a peripheral seal sealing the contents therein. The peripheral seal may be a sterile seal.

The pouch may further comprise a fluid reservoir comprising a wetting fluid.

The packaged catheter assembly of the second aspect of the invention may include any optional feature outlined above in relation to the first aspect.

According to a third aspect of the invention, there is provided an intermittent catheter assembly comprising a catheter having a proximal end for insertion into the body and a distal end, a fluid collection bag arranged to receive fluid from the distal end of the catheter, wherein the fluid collection bag comprises an outlet valve configured to control release of fluid out of the fluid collection bag in use, the outlet valve comprising: a valve base attached to a panel of the fluid collection bag, the valve base comprising a base opening for receiving a liquid output from the fluid collection bag; and an outlet element configured to be laterally rotatable relative to the valve base from a closed configuration to an open configuration.

Advantageously, the provision of the outlet element configured to be laterally rotatable relative to the valve base from a closed configuration to an open configuration means that the outlet valve is easily operable by a user. Of course the downsides of a laterally rotatable outlet element associated with Foley catheters is irrelevant to intermittent catheters as the fluid collection bag is not attached to the user. The lateral rotation of the outlet element does not require movement of the fluid collection bag to be restricted, for example by holding the bag, to operate the valve. Thus, the outlet valve can be operated by a user with limited dexterity and by users to whom the fluid collection bag is not attached to their body, such as where the catheter is an intermittent catheter.

The outlet element may be rotatable such that the outlet element moves through an arc of rotation and wherein at a first end of the arc of rotation, the outlet element may be in the closed configuration, and at a second end of the arc of rotation, the outlet element may be in the open configuration.

The fluid collection bag may comprise a base and an upper edge and first and second lateral edges. The two lateral edges may comprise a right lateral edge and a left lateral edge. The right lateral edge and left lateral edge may be defined as the right and left sides of the bag when viewing the bag with the rear panel behind the front panel, the base at the bottom of the bag and the upper edge at the top of the bag.

The outlet valve may be arranged on the front panel, rear panel, upper edge, left lateral edge, right lateral edge, or base of the fluid collection bag. The outlet valve may be arranged on the front panel or rear panel of the fluid collection bag, toward the upper edge, left lateral edge, right lateral edge, or base. The means that the outlet valve may be arranged proximal to the upper edge, left lateral edge, right lateral edge, or base. Preferably, the outlet valve is arranged on the front panel toward the base of the fluid collection bag. It will be understood that the left and right edges need not be sharp edges and whilst a preferred embodiment comprises front and rear walls joined at their periphery to define the upper edge, left lateral edge, right lateral edge and base, alternatives could be envisaged, such as a cylindrical fluid collection bag, with circular upper and lower edges and left and right edges formed by the curved left and right sides of the cylinder.

In the closed configuration, the outlet element may substantially overlie a cavity of the fluid collection bag.

The outlet element may comprise a handle.

The handle may be rotatable from a position closer to the first lateral side to a position closer to the second lateral side.

The handle may be planar. Advantageously, this means that the handle is discreet and compact, and does not protrude away from the fluid collection bag. Thus, the handle is less likely to become caught on an object or be accidentally operated by the user, which may otherwise lead to unintentional opening of the outlet element when the fluid collection bag contains fluid.

The handle may comprise a finger-hole. The handle may be sized to allow at least one finger to pass through it. The handle may be sized to allow a user to grip it between at least their thumb and index finger. The handle may be any suitable shape, for example, circular, elliptical, square or irregularly shaped. Preferably, the aperture is elliptical. Thus, the user, in particular users with limited dexterity, can more easily grasp the outlet element to operate the outlet valve.

Accordingly in one embodiment, there is provided an intermittent catheter assembly comprising a catheter having a proximal end for insertion into the body and a distal end, a fluid collection bag arranged to receive fluid from the distal end of the catheter, wherein the fluid collection bag comprises an outlet valve configured to control release of fluid out of the fluid collection bag in use, the outlet valve comprising: a valve base attached to a panel of the fluid collection bag, the valve base comprising a base opening for receiving a liquid output from the fluid collection bag; and an outlet element configured to be laterally rotatable relative to the valve base from a closed configuration to an open configuration, wherein the outlet element comprises a handle, which is operable to rotate the outlet element between the closed configuration and the open configuration, further wherein the handle is planar and comprises a finger-hole.

The handle may be operable to rotate the outlet element between the closed configuration and the open configuration. Thus, the user, in particular users with limited dexterity, can more easily grasp the outlet element to operate the outlet valve.

The plane of the handle may be aligned with, i.e., parallel with, the plane of the panel of the fluid collection bag to which the valve base is attached, when in the closed configuration. The plane of the handle may be aligned with the plane of the panel of the fluid collection bag to which the valve base is attached, when in the open configuration. Advantageously, this means that the handle provides tactile feedback to the user to confirm when the handle, and therefore the outlet element, is in the open configuration or the closed configuration as the handle will be aligned with the valve base in each configuration. The user therefore has certainty that the outlet element is either open or closed, as desired.

Moreover, there is a reduced risk of the handle being caught on an object or be accidentally operated by the user, which may otherwise lead to unintentional opening of the outlet element when the fluid collection bag contains fluid.

Accordingly in one embodiment, there is provided an intermittent catheter assembly comprising a catheter having a proximal end for insertion into the body and a distal end, a fluid collection bag arranged to receive fluid from the distal end of the catheter, wherein the fluid collection bag comprises an outlet valve configured to control release of fluid out of the fluid collection bag in use, the outlet valve comprising: a valve base attached to a panel of the fluid collection bag, the valve base comprising a base opening for receiving a liquid output from the fluid collection bag; and an outlet element configured to be laterally rotatable relative to the valve base from a closed configuration to an open configuration, wherein the outlet element comprises a handle, which is operable to rotate the outlet element between the closed configuration and the open configuration, and wherein the handle is planar and comprises a finger-hole, further wherein the plane of handle is aligned with the plane of the panel of the fluid collection bag to which the valve base is attached, when in each of the closed configuration and the open configuration.

Accordingly in one embodiment, there is provided an intermittent catheter assembly comprising a catheter having a proximal end for insertion into the body and a distal end, a fluid collection bag arranged to receive fluid from the distal end of the catheter, wherein the fluid collection bag comprises an outlet valve configured to control release of fluid out of the fluid collection bag in use, the outlet valve comprising: a valve base attached to a panel of the fluid collection bag, the valve base comprising a base opening for receiving a liquid output from the fluid collection bag; and an outlet element configured to be laterally rotatable relative to the valve base from a closed configuration to an open configuration, wherein the outlet element comprises a handle, which is operable to rotate the outlet element between the closed configuration and the open configuration, and wherein the handle is planar and extends above the outlet element.

Accordingly in one embodiment, there is provided an intermittent catheter assembly comprising a catheter having a proximal end for insertion into the body and a distal end, a fluid collection bag arranged to receive fluid from the distal end of the catheter, wherein the fluid collection bag comprises an outlet valve configured to control release of fluid out of the fluid collection bag in use, the outlet valve comprising: a valve base attached to a panel of the fluid collection bag, the valve base comprising a base opening for receiving a liquid output from the fluid collection bag; and an outlet element configured to be laterally rotatable relative to the valve base from a closed configuration to an open configuration, wherein the outlet element comprises a handle, which is operable to rotate the outlet element between the closed configuration and the open configuration, and wherein the handle is planar and extends above the valve base.

The handle may rotate by at least about 90, about 120, about 150 or about 180 degrees between the closed configuration and the open configuration. Advantageously, this means that a rotation of significant magnitude is required to operate the outlet valve whilst till providing a rotation easily manageable by those with limited dexterity. This reduces the possibility of accidentally opening the outlet valve which may occur should the required rotation be smaller.

The handle may rotate by no more than about 90, about 120, about 150 or about 180 degrees between the closed configuration and the open configuration.

The handle may be inhibited from rotating more than 180 degrees clockwise or anti-clockwise due to obstruction by the valve base.

The handle may extend above the outlet element. The handle may extend above the valve base. When in the closed or open configuration, the handle may extend laterally further than the valve base. Advantageously, this means that the handle is sufficiently sized to be easily grasped by a user, in particular by a user with limited dexterity.

The valve base may be any suitable shape, for example, circular, elliptical, square, trapezoidal or irregularly shaped. Preferably, the valve base is trapezoidal.

The valve base may be attached to a panel of the fluid collection bag any one or more of: a weld; mechanical seal; heat seal; pressure seal; adhesive; solvent bond; ultraviolet bond; ultrasonic weld; laser weld; impulse weld; or friction weld. In a preferred embodiment, the valve base is attached to a panel of the fluid collection by a weld. Advantageously, the valve base may be joined to the bag easily and added to the fluid collection bag construction as part of conventional techniques for the manufacture of fluid collection bags.

The valve base may comprise a housing which houses a portion of the outlet element and in which the outlet element rotates. Thus, the housing may protect the outlet element from knocks against other objects, or similar. This prevents the outlet element becoming damaged and potentially inoperable or faulty.

The housing may define a cylindrical bore.

The handle may be connected to the outlet element.

The handle may be integrally formed with the outlet element. The handle and the outlet element may be formed as a single moulding.

The outlet element may have a generally tubular form.

The housing may have a generally tubular form.

The outlet element have a connector portion which may be may be configured for use with a connector having a connector diameter, the housing having a diameter marginally smaller than the connector diameter such that, on insertion of the connector onto a distal end of the housing in use, the connector portion can accommodate the connector.

The connector portion may comprise a flange distal to the outlet element. The flange may be arranged to inhibit a connector being extended toward the outlet element whilst still allowing the connector portion to accommodate the connector. The flange may be annular. The flange may be tapered inwardly as it extends away from the outlet element.

The valve base may be formed of a material which is more rigid than the relatively flexible material from which the fluid collection bag is formed. This assists the user in rotating the outlet element.

The valve base, housing and/or outlet element may be formed, for example, from polypropylene or High-density polyethylene (HDPE), acrylonitrile butadiene styrene (ABS) or polycarbonate. The materials of the valve base, housing and the outlet element may be chosen to have different shrinkage rates to allow for hot assembly of these two components to produce a good seal therefore preventing leakage. The use of different materials may also be different to avoid sticking between the two components over time.

The outlet element may be rotatable about a vertical axis of rotation when in use.

The outlet element may be rotatable about an axis parallel to the longest dimension of the fluid collection bag. The longest dimension of the fluid collection bag may be along the left lateral edge and along the right lateral edge, i.e., between the upper edge and the base.

The outlet element may be rotatable about an axis parallel to the plane of the panel of the fluid collection bag to which the valve base is attached, when in the closed configuration or the open configuration.

The valve base may be arranged on the front panel of the fluid collection bag. The outlet element may be rotatable about an axis parallel to the plane of a front panel of the fluid collection bag. The valve base may be arranged on the rear panel of the fluid collection bag. The outlet element may be rotatable about an axis parallel to the plane of a rear panel of the fluid collection bag.

The outlet valve may be arranged on the front panel or on the rear panel of the fluid collection bag.

The outlet element may protrude from an outer face of the valve base. The outlet element may protrude from an outer face of the valve base no more than 1 cm, 1.5 cm, 2 cm, 2.5 cm, 3 cm, 3.5 cm, 4 cm, 4.5 cm, or no more than 5 cm. The outlet element may protrude from an outer face of the valve base by at least 1 cm, 1.5 cm, 2 cm, 2.5 cm, 3 cm, 3.5 cm, 4 cm, 4.5 cm, or at least 5 cm.

When in the open configuration or in the closed configuration, the outlet valve may extend from the surface of the fluid collection bag no more than 1 cm, 1.5 cm, 2 cm, 2.5 cm, 3 cm, 3.5 cm, 4 cm, 4.5 cm, or no more than 5 cm.

When in the open configuration or in the closed configuration, the outlet valve may extend from the surface of the fluid collection bag by at least 0.5 cm, 1 cm, 1.5 cm, 2 cm, 2.5 cm, 3 cm, 3.5 cm, 4 cm, 4.5 cm, or at least 5 cm.

Advantageously, this provides a discreet and compact outlet valve which is not prone to accidental operating of the valve.

The catheter may be a urinary catheter. The catheter may be a male urinary catheter (i.e. a catheter for catheterisation of men). The catheter may be a female urinary catheter (i.e. a catheter for catheterisation of women). The catheter may be an intermittent catheter. In one embodiment, the catheter is an intermittent male urinary catheter. The features of the present invention allow intermittent urinary catheters to be used more easily and effectively.

The catheter may have a length of at least about 10 cm, about 15 cm, about 20 cm, about 25 cm, about 30 cm, about 35 cm, about 40 cm, about 45 cm, about 50 cm, about 55 cm, about 60 cm, about 65 cm, about 70 cm, about 80 cm, about 90 cm, or at least about 100 cm.

The catheter may have a width of no more than about 10 cm, about 15 cm, about 20 cm, about 25 cm, about 30 cm, about 35 cm, about 40 cm, about 45 cm, about 50 cm, about 55 cm, about 60 cm, about 65 cm, about 70 cm, about 80 cm, about 90 cm, or no more than about 100 cm.

At least a portion of the fluid collection bag may be formed from an opaque material or be coated to present the appearance of being opaque. At least 10%, 20%, 30%, 40%, 50%, 60%, 70% or at least 80% of the fluid collection bag may be formed from an opaque material or be coated to present the appearance of being opaque. No more than 10%, 20%, 30%, 40%, 50%, 60%, 70% no more than 80% of the fluid collection bag may be formed from an opaque material or be coated to present the appearance of being opaque. The fluid collection bag may be opaque at the inlet and/or outlet. Advantageously, this makes the contents of the pouch during and after use at least partially hidden and, therefore, more discrete for the user.

The outlet valve may be formed of opaque materials.

The fluid collection bag may comprise a front panel and a rear panel.

The outlet valve may be arranged off-centre when the fluid collection bag is viewed with the rear panel behind the front panel.

Accordingly in one embodiment, there is provided an intermittent catheter assembly comprising a catheter having a proximal end for insertion into the body and a distal end, a fluid collection bag arranged to receive fluid from the distal end of the catheter, wherein the fluid collection bag comprises a front panel and a rear panel, wherein the fluid collection bag further comprises an outlet valve configured to control release of fluid out of the fluid collection bag in use, the outlet valve comprising: a valve base attached to the front panel of the fluid collection bag, the valve base comprising a base opening for receiving a liquid output from the fluid collection bag; and an outlet element configured to be laterally rotatable relative to the valve base from a closed configuration to an open configuration, further wherein the outlet valve is arranged off-centre when the fluid collection bag is viewed with the rear panel behind the front panel.

The outlet valve may be arranged off-centre, toward the left lateral edge or toward the right lateral edge.

Accordingly in one embodiment, there is provided an intermittent catheter assembly comprising a catheter having a proximal end for insertion into the body and a distal end, a fluid collection bag arranged to receive fluid from the distal end of the catheter, wherein the fluid collection bag comprises a front panel and a rear panel, wherein the fluid collection bag further comprises an outlet valve configured to control release of fluid out of the fluid collection bag in use, the outlet valve comprising: a valve base attached to the front panel of the fluid collection bag, the valve base comprising a base opening for receiving a liquid output from the fluid collection bag; and an outlet element configured to be laterally rotatable relative to the valve base from a closed configuration to an open configuration, further wherein the outlet valve is arranged off-centre, toward a left lateral edge or toward a right lateral edge of the fluid collection bag.

The outlet valve may be arranged off-centre toward the side of the fluid collection bag on which the hand strap is arranged. For example, the hand strap may be arranged on the corner where the upper edge and right lateral edge meet, and the outlet valve may be arranged proximal to the base, off-centre toward the right lateral edge. The hand strap may arranged toward the first lateral edge or toward the right lateral edge of the fluid collection bag. The outlet valve may be arranged off-centre toward the lateral edge of the fluid collection bag toward which the hand strap is arranged.

Accordingly in one embodiment, there is provided an intermittent catheter assembly comprising a catheter having a proximal end for insertion into the body and a distal end, a fluid collection bag arranged to receive fluid from the distal end of the catheter, wherein the fluid collection bag further comprises an outlet valve configured to control release of fluid out of the fluid collection bag in use, the outlet valve comprising: a valve base attached to a panel of the fluid collection bag, the valve base comprising a base opening for receiving a liquid output from the fluid collection bag; and an outlet element configured to be laterally rotatable relative to the valve base from a closed configuration to an open configuration, wherein the fluid collection bag comprises a hand strap arranged toward a left lateral edge or a right lateral edge of the fluid collection bag, and the outlet valve is arranged off centre toward the edge of the fluid collection bag on which the hand strap is arranged.

The fluid collection bag may comprise a bond between the two panels defining an internal cavity.

According to a fourth aspect of the invention, there is provided a packaged catheter assembly comprising the intermittent catheter assembly of the third aspect, wherein the intermittent catheter assembly is arranged within a pouch.

The pouch may be sealed by a peripheral seal sealing the contents therein. The peripheral seal may be a sterile seal.

The pouch may further comprise a fluid reservoir comprising a wetting fluid.

The packaged catheter assembly of the fourth aspect of the invention may include any optional feature outlined above in relation to the third aspect.

In broad terms, the present invention concerns a catheter assembly comprising a catheter.

According to a fifth aspect of the present invention there is provided a catheter assembly comprising a catheter having a proximal end for insertion into the body and a distal end, a fluid collection bag arranged to receive fluid from the distal end of the catheter, a sleeve enclosing the catheter along the length of the catheter from the proximal end to the distal end, and a fluid reservoir arranged at the proximal end, wherein the fluid reservoir is configured to release wetting fluid into the sleeve to wet the catheter and wherein the sleeve is in fluid communication with the fluid collection bag. Consequently, excess wetting fluid may flow from the sleeve into the fluid collection bag.

Advantageously, the present invention provides for a catheter assembly which benefits from the introduction of wetting fluid at the proximal end of the catheter which reduces the risk of discomfort and injury while using the catheter as the proximal end, which enters the body first, is adequately wetted prior to use. In addition, as the wetting fluid is contained within the sleeve and flows from the proximal end to the distal end, the fluid is used efficiently to wet the whole length of the catheter by prioritising the parts of the catheter that enter the body first. Due to the sleeve being in fluid communication with the fluid collection bag, wetting fluid cannot escape outside the sleeve and as such the user's hands or clothing does not inadvertently contact the wetting fluid which could make handling the catheter difficult. This ensures that the catheter is easy and convenient to use, as well as being discrete and hygienic to dispose of as the wetting fluid is contained within the sleeve/bag.

The catheter may be a urinary catheter. The catheter may be a male urinary catheter. The catheter may be a female urinary catheter. The catheter may be an intermittent catheter. In one embodiment, the catheter is an intermittent male urinary catheter. Thus, the features of the present invention allow intermittent male urinary catheters to be adequately wetted prior to use which can be more difficult than for other types of catheters which are generally shorter.

The sleeve may comprise a flexible plastics material. The sleeve may be liquid impermeable. The sleeve may comprise a thermoplastic polyurethane (TPU) or low-density polyethylene (LDPE). Thus the sleeve is cheap and easy to produce and can be easily manipulated by the user during use.

The catheter may comprise a proximal end for insertion into the body and a distal end. The sleeve may comprise a proximal end and a distal end. The distal end of the sleeve may be secured or attached either directly or indirectly to the distal end of the catheter. The sleeve may be configured to enclose a majority of the length of the catheter. The sleeve may be liquid permeable. Where the sleeve is liquid permeable, it may be provided in a configuration in which it covers a majority of the length of the catheter.

The fluid reservoir may be configured to retain wetting fluid within it. The fluid reservoir may comprise a deformable, frangible or burstable sachet. A deformable, frangible or burstable reservoir enhances the convenience for a user as fluid can be easily released without additional tools. A sachet in particular can be more easily handled on an assembly line compared to handling fluid itself.

The wetting fluid may activate the surface of the catheter and render it lubricious. The wetting fluid may be water, such as where the catheter has a hydrophilic surface. Any suitable wetting fluid may be used, for example polar (e.g. water-based) or non-polar (e.g. oil-based) wetting fluids depending on the surface properties of the catheter. The fluid reservoir may be configured to retain at least 5, 10, 15, 25, or 50 ml of wetting fluid.

The fluid collection bag may be any suitable shape or size, for example rectangular, circular, elliptical, cuboid, spherical, etc. The fluid collection bag may comprise a front panel and a rear panel. The fluid collection bag may comprise a peripheral bond joining the periphery of the front panel and rear panel to form the fluid collection bag. The peripheral bond may define a base, two lateral edges and an upper edge of the fluid collection bag. The two lateral edges may comprise a right lateral edge and a left lateral edge. The right lateral edge and left lateral edge may be defined as the right and left sides of the bag when viewing the bag with the rear panel behind the front panel, the base at the bottom of the bag and the upper edge at the top of the bag. The bag may have a width between the left lateral edge and right lateral edge of at least 10, 12, 15 or 20 cm. The bag may have a width no more than 25, 20, or 15 cm. Preferably, the width is between 10 and 20 cm, most preferably between 12 and 15 cm. The base may define a bottom of the bag. The upper edge may define a top of the bag. The height of the bag from the base to the upper edge may be at least 25, 30, 35 or 40 cm. The height of the bag may be no more than 45, 40, 35 or 30 cm. The height may be between 25 and 40 cm, 30 and 35 cm, or most preferably 33 and 35 cm. The bag may be configured to hold at least 500, 700, or 1000 ml of fluid, and may hold no more than 1000 ml, for example, it may hold at least 700 ml of fluid.

The fluid collection bag may comprise a flexible plastics material. For example, polypropylene (PP), polyethylene terephthalate (PET), low density polyethylene (LDPE), metalized polyester (MET PET), orientated polypropylene (OPP), polyvinylpyrrolidone (PVP), or polyvinyl chloride (PVC).

The peripheral bond may provide a water-tight seal. Preferably, the peripheral bond provides a sterile seal. The peripheral bond may comprise any one or more of: a weld; mechanical seal; heat seal; pressure seal; adhesive; solvent bond; ultraviolet bond; ultrasonic weld; laser weld; impulse weld; or friction weld. This ensures that the catheter is maintained within a sterile environment prior to use and also that once the catheter has been used any fluid contained within the bag does not leak out unintentionally.

The fluid collection bag may comprise an inlet to allow fluid to enter the bag from the distal end of the catheter and optionally via the sleeve. The inlet may be connected to one end of the sleeve which corresponds to the distal end of the catheter. The inlet may comprise a one-way valve which prevents fluid flow through the inlet from the fluid collection bag to the sleeve and/or catheter. Thus, the inlet allows liquid to pass into the fluid collection bag but optionally may prevent fluid flow out of the bag and into the sleeve. This can reduce the likelihood of leaking.

The catheter assembly may be configured to allow fluid to pass directly from the sleeve to the inlet and/or directly from the catheter to the inlet. One lateral side (e.g. a right lateral side) of the catheter may be attached to one side (e.g. a right side) of the inlet. There may be a gap between a different lateral side (e.g. a left lateral side) of the catheter and a respective different side (e.g. a left side) of the inlet. The distal end of the catheter may be within the inlet. The sleeve may be sealed around a periphery of the inlet. As such, fluid travelling within the sleeve is directed into the inlet directly via the gap. In addition, fluid travelling within the catheter passes out the distal end and into the inlet. In other examples, the catheter may be positioned differently and may be attached, for example, to the left side of the inlet.

The catheter assembly may be configured to allow fluid to pass from the catheter into the sleeve. One lateral side (e.g. a right lateral side) of the catheter may be attached to a side (e.g. right side) of the sleeve. A distal end of the catheter may be within the sleeve. A distal end of the catheter may not be within the inlet. The sleeve may be sealed around a periphery of the inlet. As such, fluid travelling within the sleeve is directed into the inlet directly and fluid travelling within the catheter passes out the distal end into the sleeve and then from there into the inlet. In other examples, the catheter may be positioned differently and may be attached to a left side of the sleeve (or indeed a front side or rear side). Furthermore, the distal end of the catheter may be blocked and an outlet may be provided in a lateral side of the catheter to allow fluid to pass out into the sleeve.

The catheter assembly may be configured to allow fluid to pass from the sleeve into the catheter. One lateral side (e.g. a right lateral side) of the catheter may be attached to one side (e.g. a right side) of the inlet. The inlet may be sealed around the catheter on all sides. Consequently, there may be no gap between the inlet and the catheter. As such, fluid may only pass from the sleeve/catheter into the inlet if it is within the catheter. A side, e.g. a lateral side of the catheter may comprise a sleeve intake opening. The sleeve intake opening may be any suitable shape such as oval or circular. The sleeve intake opening may be adjacent to the inlet. The sleeve intake opening may permit the passage of fluid from the sleeve into the catheter. Thus fluid may enter the catheter from the sleeve to join fluid travelling within the catheter. From here, the fluid may pass out the distal end of the catheter and into the inlet. Many other configurations may be possible to achieve the same effect, for example, the sleeve may be attached to the outer sides of the catheter itself.

The inlet of the fluid collection bag may be positioned at a corner of the peripheral bond. The fluid collection bag may comprise a neck adjacent to the inlet. The neck may comprise one or more projections extending into the fluid collection bag. The one or more projections may comprise regions of the peripheral bond that extend into the bag. The one or more projections may restrict fluid flow through a region of the bag adjacent to the inlet. The projections may be configured to provide gripping points adjacent to the inlet. Thus, the fluid collection bag resists the flow of fluid back towards the inlet and also is more easily handled by the user.

Any one or more of the one or more projections may have any one or more of the features of a projection as described below.

A projection may have any suitable size or shape. A projection may be triangular, square, rectangular, circular, ellipsoidal or irregularly shaped, preferably, a projection is triangular. A point on the projection furthest from an edge of the bag may be a (right-angled) corner in the projection. The projection may be a right-angled triangle. The hypotenuse of the projection may be aligned parallel to an edge of the bag. Thus, the projections can be suitably shaped to minimise loss of volume in the bag due to the projections. In addition, its shape can help to direct fluid flow within the bag, for example assisting flow into the bag and reducing flow out of the bag.

A projection may extend along no more than 90%, 80%, 70%, 60%, 50%, 40%, 30%, 20% or 10% of the length of an edge of the bag. A projection may extend by at least 10%, 20%, 30%, 40%, 50%, 60%, 70%, 80%, or 90% of the length of an edge of the bag. A projection may comprise a corner of the bag. A projection may not comprise from a corner of the bag. Thus, the projections can be flexibly sized and positioned to be most effective.

A projection may be a lateral projection extending from a left (or right) lateral edge of the bag. The lateral projection may be provided on the same side of the bag as the inlet. The lateral projection may extend along 10-40% of the length of the lateral edge of the bag, preferably 25%. The lateral projection may extend across 5-20% of the width of the bag, preferably 10%. The lateral projection may be a right-angled triangle. The right-angled vertex of the lateral projection may project into the bag. The right-angled vertex may be positioned below the inlet inside the bag. The right-angled vertex may be positioned 10% of the way from the upper edge to the base of the bag. The non-right-angled vertices of the lateral projection may be provided on the lateral edge of the bag from which the lateral projection extends. Thus, the lateral projection can assist in directing the flow of fluid into the bag and also helps prevent fluid flowing directly from the bag back up into the sleeve/catheter.

A projection may be an end projection extending from the upper edge or base of the bag. Preferably, the end projection extends from the upper edge. The end projection may be provided adjacent to the inlet. The end projection may extend along 40-95% of the width of the bag, preferably 90%. The end projection may extend from a corner of the bag. The end projection may extend down 5-20% of the length of the bag, preferably 10%. The end projection may be a right-angled triangle. The right-angled vertex of the end projection may project into the bag. The right-angled vertex may be positioned adjacent the inlet inside the bag. The right-angled vertex may be positioned 20% of the way across the width of the bag. The non-right-angled vertices of the lateral projection may be provided on the edge of the bag from which the end projection extends. Thus, the end projection can assist in directing the flow of fluid into the bag and also helps prevent fluid flowing directly from the bag back up into the sleeve/catheter.

In a preferred embodiment, the fluid collection bag comprises both a lateral projection and an end projection extending from the upper edge of the bag as described above. Thus, the two projections work together to assist the user in handling the bag and also to manage fluid flow into/out of the bag.

A projection may comprise a rib. The rib may resist bending or deformation in a given direction. The rib may facilitate folding of the fluid collection bag. The rib may assist the user in grasping or gripping the bag. The strengthening element may comprise a region of the projection that is thicker or thinner than the rest of the projection. The rib may be any suitable shape or size, such as a line which could be straight or curved. The rib may extend across the projection. The rib may extend from an edge of the bag. The rib may extend from a midpoint of the projection along the edge of the bag. The rib may extend to a point on the projection furthest from the edge of the bag, for example a right-angled vertex of a right-angled projection. The rib may be at an angle (i.e. not parallel or perpendicular to) an edge of the bag. Preferably, the lateral projection may comprise a rib. Thus, the mechanical properties of the projection can be controlled through use of a rib to the benefit of the user.

A projection may be a join that is independent from the peripheral bond of the fluid collection bag. A join may have any one or more of the features of a projection as described herein. A join can be beneficial to reduce the total area of the panels of the fluid collection bag that are sealed together.

The fluid collection bag may comprise a finger hole. The finger hole may be sized to allow a user to put their finger through the finger hole and handle the bag. Thus, the finger hole allows the bag to be handled more easily by a user. The finger hole may be provided at an edge of the bag. The finger hole may be provided in the peripheral bond of the bag. The finger hole may be provided at a corner of the bag. The finger hole may be provided at any of the following corners: where the left lateral edge meets the base; where the base meets the right lateral edge; where the right lateral edge meets the upper edge; or where the upper edge meets the left lateral edge. Where the finger hole is provided in the bag, the peripheral bond may be shaped to accommodate the finger hole. The peripheral bond may truncate a corner of the bag to accommodate the finger hole. The peripheral bond may project into the bag to accommodate the finger hole. As an alternative to the finger hole, the fluid collection bag may comprise a series of ridges or otherwise easily gripped surface or feature in the place of the finger hole to allow the bag to be easily handled.

The fluid collection bag may comprise more than one finger hole, for example at least two, three or more finger holes. One, two, three or more, or each finger hole may have any one or more of the features of the finger hole described above. Thus, easy handling of the bag is facilitated through positioning of finger holes or equivalent features which also does not restrict or affect the volume of liquid that can be contained by the bag.

The finger hole may be provided in a projection of the fluid collection bag. Preferably, the end projection as described above comprises a finger hole. The finger hole may be positioned adjacent to the right-angled vertex of the end projection.

The one or more finger holes may be configured to allow the bag to be rotated to a horizontal position wherein the base of the bag is aligned vertically. In a preferred embodiment, the fluid collection bag comprises a finger hole at either end of the right lateral edge, and/or a finger hole at either end of the left lateral edge, and/or a finger hole along the upper edge and a finger hole along the base. Advantageously, this allows the bag to be rotated in a controlled manner which aids with handling and using the bag.

The fluid collection bag may comprise a fill level indicator. The fill level indicator may comprise fill markers. The fill markers may allow the user to measure the volume of fluid contained within the fluid collection bag. The fill markers may be regularly spaced or irregularly spaced. The fill markers may correspond to an absolute volume of liquid contained within the bag. The fill markers may correspond to a proportion of the volume of the bag which is filled with fluid. The fill markers may indicate when a safe fill level of the bag has been reached. The fill level indicator may be positioned in a transparent or translucent region of the bag. Thus, the fill level indicator ensures the bag is not overfilled.

The sleeve may comprise a reservoir adapter. An end of the sleeve may comprise a reservoir adapter. The reservoir adapter may be configured to engage the fluid reservoir. The reservoir adapter may be configured to receive wetting fluid from the fluid reservoir. The reservoir adapter may be configured to detach from the fluid reservoir. The reservoir adapter may be configured to permit the catheter to pass through the reservoir adapter.

According to a broad aspect of the present invention, there is provided catheter assembly comprising a fluid reservoir, a catheter which comprises a proximal end for insertion into the body and a distal end, and a sleeve extending from the proximal end to the distal end, wherein an end of the sleeve comprises a reservoir adapter configured to: engage the fluid reservoir; receive a wetting fluid from the fluid reservoir into the sleeve to wet the catheter; and detach from the fluid reservoir to permit the catheter to pass through the reservoir adapter.

The catheter assembly may be a packaged catheter assembly comprising a pouch containing the fluid reservoir and catheter. According to a sixth aspect of the present invention, there is provided a packaged catheter assembly comprising a fluid reservoir, a catheter, and a pouch containing the fluid reservoir and catheter, wherein the catheter comprises a proximal end for insertion into the body, a distal end, and a sleeve extending from the proximal end to the distal end, wherein an end of the sleeve comprises a reservoir adapter configured to: engage the fluid reservoir; receive a wetting fluid from the fluid reservoir into the sleeve to wet the catheter; and detach from the fluid reservoir to permit the catheter to pass through the reservoir adapter.

Thus, wetting fluid is efficiently and easily introduced to wet the catheter as the reservoir adapter retains the fluid reservoir at one end of the sleeve. This ensures that wetting fluid is not wasted and also that wetting fluid is less likely to leak out and wet the external surface of the sleeve or pouch which may cause discomfort for the user.

The sleeve may comprise a proximal end and a distal end corresponding to the proximal end and distal end of the catheter respectively. The proximal end of the sleeve may comprise the reservoir adapter. The reservoir adapter may be positioned at or adjacent to the proximal end of the catheter. Thus, the reservoir is adapted to deliver wetting fluid to the proximal end of the catheter first, this ensures that the part of the catheter first introduced into the body is most likely to be adequately wetted. This reduces the risk of injury or discomfort during use of the catheter.

The reservoir adapter may tubular. The reservoir adapter may comprise a bore therethrough. The bore may be sized to allow the catheter to pass through the reservoir adapter. The bore may be sized to allow the catheter and wetting fluid to pass through the reservoir adapter. The reservoir adapter may be configured to inhibit passage of the catheter through the reservoir adapter/bore while the fluid reservoir is engaged by the reservoir adapter. The fluid reservoir may be configured to block the catheter from passing through the reservoir adapter/bore. The fluid reservoir may be configured to block the catheter from passing through the reservoir adapter/bore if the fluid reservoir is engaged by the reservoir adapter. Thus, the catheter may be withdrawn from the sleeve through the reservoir adapter and bore, but only once the fluid reservoir is removed from the reservoir adapter. This helps to protect the catheter from dirt and damage prior to use.

The reservoir adapter may comprise a distal end corresponding to an end of the reservoir adapter attached to the sleeve. The distal end may be circular or cylindrical. The distal end may have an annular cross-section. The distal end may comprise an internal diameter sized to allow the catheter and wetting fluid to pass therethrough. The distal end may have an outer structure that is any shape and optionally does not correspond to the shape of the inner structure. Thus, the sleeve and catheter are attached to the reservoir adapter and the catheter may pass through it.

The reservoir adapter may be configured to engage the fluid reservoir by any suitable means, such as push-fit, bayonet fit, or screw-fit engagement. The reservoir adapter may have an internal size and shape that matches the fluid reservoir. Thus, the fluid reservoir may be easily push-fit into the reservoir adapter.

The fluid reservoir may be a sachet. The fluid reservoir may be any suitable shape or size, such as square, rectangular, circular, ellipsoidal or irregularly shaped. Preferably, the fluid reservoir is elongate and optionally rectangular. The reservoir adapter may be configured to engage a first end of the fluid reservoir. The fluid reservoir may comprise a second end distal the first end. The fluid reservoir may have a length defined as the distance between the first end and the second end. The length of the fluid reservoir may be at least 50 mm, at least 60 mm, at least 70 mm, at least 80 mm, at least 90 mm, at least 100 mm, at least 120 mm or at least 140 mm. the length of the fluid reservoir may be no more than 160, 150, 140, 120, 100, 90 or 80 mm. The length of the fluid reservoir may be between 120 and 150 mm, e.g. 140 mm, which is typical for fluid reservoirs used for male intermittent catheters. Alternatively the length of the fluid catheter may be between 70 and 90 mm, e.g. 80 mm. This length is typical for female intermittent catheters, but, because of the provision of the reservoir adaptor, which ensures that wetting fluid is not wasted, may be suitable even for male catheters (which are much longer than female catheters).

The reservoir adapter may be configured to extend around the first end of the fluid reservoir. The reservoir adapter may be configured to extend along the fluid reservoir. The reservoir adapter may be configured to extend along at least 10%, 20%, 30%, 40% or 50% of the length of the fluid reservoir. The reservoir adapter may be configured to extend along no more than 60%, 50%, 40%, 30% or 20% of the length of the fluid reservoir. Preferably, the reservoir adapter is configured to extend along a third of the length of the fluid reservoir. Thus the reservoir adapter ensures that the fluid reservoir is securely engaged and that wetting fluid is delivered efficiently from the fluid reservoir into the sleeve.

The reservoir adapter may comprise a proximal end distal from the sleeve. The proximal end may be configured to receive and/or retain the fluid reservoir. The proximal end may extend around and along the fluid reservoir as described above. Where the fluid reservoir is a rectangular sachet, the proximal end may comprise an elliptical annular cross-section. Thus, the fluid reservoir is engaged in a push-fit/interference fit with the reservoir adapter.

The reservoir adapter may comprise a midsection between the proximal end and distal end. The proximal end may be tapered, for example where the proximal end and distal end are different shapes/sizes. The proximal end may have a larger diameter than the distal end. The midsection may be configured to funnel wetting fluid towards the proximal end of the reservoir adapter. The midsection may comprise a convex surface. Thus, the midsection aids handling of the reservoir adapter by the user and also helps ensure wetting fluid passes through the reservoir adapter onto the catheter.

The reservoir adapter may comprise a handling element. The handling element may be configured to allow the user to move at least part of the sleeve along the catheter, preferably the proximal end of the sleeve. The reservoir adapter/handling element may have a higher rigidity than the sleeve and/or catheter. The reservoir adapter/handling element may comprise high density polyethylene (HDPE). Thus, the reservoir adapter may act as a handling element for the catheter and provide the user with a convenient point to control the use of the catheter and the movement of the sleeve up and down the catheter.

The reservoir adapter may comprise an inserter tip. The inserter tip may disposed at the proximal end of the reservoir adapter distal from the sleeve. The inserter tip may be configured to guide the passage of the catheter into the body. Thus, the reservoir adapter can be used by the user to ensure comfortable, safe and efficient use of the catheter.

The fluid reservoir may comprise an activation marker. The fluid reservoir may be configured to release wetting fluid upon activation of the activation marker. The activation marker may be on a surface of the fluid reservoir. The activation marker may comprise a region of the fluid reservoir that is identifiable to the user, for example through visual and/or tactile feedback. The activation marker may be located at a position on the fluid reservoir that is not enclosed by the reservoir adapter when the fluid reservoir is engaged by the reservoir adapter. Preferably, the activation marker may be at least visually recognisable, for example a different colour, or pattern, to the fluid reservoir. Thus, the activation marker is easily recognisable while the fluid reservoir is engaged by the reservoir adapter.

The fluid reservoir may be configured to break to release wetting fluid, for example by creating a tear in the fluid reservoir. The fluid reservoir may be configured to break at a point enclosed by the reservoir adapter when the fluid reservoir is engaged by the reservoir adapter. The reservoir adapter and/or fluid reservoir may comprise a sealing element. The sealing element may be configured to prevent wetting fluid from passing out of the proximal end of the reservoir adapter. The sealing element may be configured to provide resistance to movement of the reservoir adapter with respect to the fluid reservoir. The sealing element may comprise an O-ring provided on an internal surface of the reservoir adapter and/or an O-ring provided on an external surface of the fluid reservoir. Thus, wetting fluid is released into the reservoir adapter and does not leak out of the distal end of the reservoir adapter where it could cause discomfort/inconvenience for the user.

The fluid reservoir may be secured to the pouch, e.g. secured by adhesive, by a weld, or by other means. As such, the fluid reservoir may be retained in the pouch after wetting, preventing misuse, e.g. by users taking the fluid reservoir out, pulling back the sleeve and seeking to wet the catheter manually.

The catheter assembly may comprise a jacket surrounding the fluid reservoir. The jacket may be configured to receive a wetting fluid from the reservoir. The jacket may be configured to direct wetting fluid into the sleeve to wet the catheter. Consequently, in a broad aspect of the invention there is provided a catheter assembly comprising a fluid reservoir and a catheter, the catheter having a proximal end for insertion into the body, a distal end, and a sleeve extending from the proximal end to the distal end; the assembly further comprising a jacket surrounding the fluid reservoir, the jacket being configured to receive a wetting fluid from the reservoir and direct the wetting fluid into the sleeve to wet the catheter.

The wetting fluid may be directed to the proximal end of the catheter before the distal end of the catheter. The wetting fluid may be directed into the proximal end of the sleeve. Thus, according to a seventh aspect of the invention, there is provided a catheter assembly comprising a fluid reservoir and a catheter, the catheter having a proximal end for insertion into the body, a distal end, and a sleeve extending from the proximal end to the distal end; the assembly further comprising a jacket surrounding the fluid reservoir, the jacket being configured to receive a wetting fluid from the fluid reservoir and direct the wetting fluid into the sleeve at the proximal end to wet the catheter.

Thus, the provision of a jacket around the fluid reservoir ensures that the wetting fluid released by the reservoir is directed efficiently onto the catheter to wet the catheter. This prevents loss/leaks of wetting fluid which can lead to insufficient wetting of the catheter rendering it unsuitable for use and/or causing discomfort/injury to the user during use.

The jacket also allows more design freedom in relation to the fluid reservoir and catheter assembly as a whole as the jacket controls the delivery of fluid to the sleeve/catheter. This allows the fluid reservoir/catheter assembly to be more user friendly.

The reservoir adapter may be configured to engage the jacket. The reservoir adapter may engage the fluid reservoir via the jacket. The reservoir adapter may be configured to receive wetting fluid from the fluid reservoir via the jacket. The jacket may be configured to block the catheter from passing through the reservoir adapter (until the jacket is detached from the reservoir adapter). The reservoir adapter may be configured to detach from the jacket (to allow the catheter to pass through the reservoir adapter). Thus, the jacket can provide a more flexible engagement between the reservoir adapter and fluid reservoir, for example, where the fluid reservoir is an inappropriate size to directly engage with the reservoir adapter. The jacket may comprise a wetting aperture. The wetting aperture may be arranged at a position corresponding to a part of the fluid reservoir that is enclosed by the reservoir adapter. The wetting aperture may be arranged at an end of the fluid reservoir, preferably the first end referred to above. The wetting aperture may be configured to direct wetting fluid into the sleeve and/or onto the catheter. Thus, the wetting aperture helps ensure that wetting fluid released by the fluid reservoir is directed into the sleeve.

The fluid reservoir may be configured to rupture before the jacket. The jacket may be configured not to rupture under compression. The wetting aperture may be configured to allow fluids to exit and enter the jacket. Thus, the jacket remains intact when the user pinches the fluid reservoir to rupture it as any pressure build up is relieved through the wetting aperture.

The jacket may be impermeable to fluids, for example impermeable to liquid, in particular impermeable to water. The jacket may be formed of a material that is impermeable to fluids. The jacket may be formed from a flexible plastics material, for example a thermoplastic polyurethane (TPU) or low-density polyethylene (LDPE). Thus, the jacket can ensure that wetting fluid is directed into the sleeve and doesn't escape/leak out.

The jacket may be formed of a transparent or translucent material. The jacket may be formed of an opaque material. The jacket may comprise a window of transparent or translucent material at a position corresponding to the activation marker of the fluid reservoir. The jacket may comprise an activation marker. The activation marker may be on a surface of the jacket. The activation marker may comprise a region of the jacket that is identifiable to the user, for example through visual and/or tactile feedback. The activation marker may be located at a position on the jacket corresponding to a position of the fluid reservoir which when pressed will cause the reservoir to rupture. The activation marker may be in a position corresponding to the activation marker positioned on the fluid reservoir. Thus, the user can see the activation marker of the fluid reservoir through the jacket (through a window in the jacket if necessary), or alternatively can activate the fluid reservoir through an activation marker disposed on the jacket.

The jacket may be formed from two strips of material joined about their edges. Each strip of material may have a shape corresponding to the shape of the fluid reservoir. The wetting aperture may comprise a section around the edge of the jacket where the two strips of material are not joined together. The wetting aperture may comprise a section of the jacket which has been cut or punched to form the wetting aperture. Thus, the jacket and wetting aperture can be easily formed around the fluid reservoir if required.

The jacket may be secured to the pouch, e.g. secured by adhesive, by a weld, or by other means. As such, the jacket and the fluid reservoir within may be retained in the pouch after wetting, preventing misuse, e.g. by users taking the fluid reservoir out, pulling back the sleeve and seeking to wet the catheter manually.

In broad terms the invention also concerns a packaged catheter assembly comprising a pouch and the catheter assembly, wherein the pouch is configured to contain the catheter assembly within it. The fluid collection bag may be arranged within the pouch. The pouch may comprise an interaction region to form an opening in the pouch and allow the catheter and fluid collection bag to be removed from the pouch. The fluid collection bag may be arranged adjacent to the opening.

Thus, according to a eighth aspect of the present invention there is provided a packaged catheter assembly comprising a catheter, a pouch, and a fluid collection bag configured to receive fluid from the catheter in use, wherein: the catheter and fluid collection bag are arranged within the pouch; the pouch comprises an interaction region to form an opening in the pouch and allow the fluid collection bag and catheter to be removed from the pouch; and the fluid collection bag is arranged adjacent to the opening.

With this arrangement, the fluid collection bag protects the catheter from coming into contact with dirt as the pouch is opened and also helps prevent the user from accidentally touching the catheter during opening of the pouch. This helps to reduce risk of infection during use of the pouch as the catheter is less likely to be dirty.

The fluid collection bag may be provided in a stowed configuration, preferably while inside the pouch. The fluid collection bag may be configured to remain in the stowed configuration. In the stowed configuration, the fluid collection bag may be folded or rolled to reduce its size in any one dimension. The stowed configuration may be a folded or rolled configuration. In the stowed configuration, the fluid collection bag may be cylindrical in shape. Thus, the size of the fluid collection bag is minimised inside the pouch, and this assists the user in removing the bag and other elements of the packaged catheter assembly from the pouch. The stowed configuration of the fluid collection bag further inhibits fluid flow onto the exterior surface of the fluid collection bag as it must penetrate between folds.

The pouch may comprise a flexible plastics material. For example, polypropylene (PP), polyethylene terephthalate (PET), low density polyethylene (LDPE), metalized polyester (MET PET) or orientated polypropylene (OPP).

The pouch may comprise two walls (a front wall and a rear wall) and a peripheral seal between the two walls. The peripheral seal may provide a sterile seal. Thus, the pouch may maintain the sterility of its contents.

The peripheral seal may form one or more edges of the pouch. The peripheral seal may comprise a left lateral edge, right lateral edge, an upper edge and a base of the pouch. The left and right lateral edges may be defined as the left and right edges when viewing the pouch with the front wall in front of the rear wall, the base at the bottom and the upper edge at the top. The pouch may be any suitable shape such as circular, elliptical, triangular, tubular, prismatic or polygon, preferably the pouch is rectangular. The pouch may have a width defined as the distance between the left lateral edge and right lateral edge of the pouch, the width preferably being at least 60, 70, 80, 90 or 100 mm. The pouch may have a width of no more than 110, 100, 90, 80, or 70 mm. Preferably, the width is between 90 and 110 mm, and most preferably about 100 mm. The pouch may have a height defined as the distance between the upper edge and base, the height preferably being at least 100, 125, 150, 175, 200 or 225 mm. The pouch may have a height of no more than 250, 225, 200, 175, 150, or 125 mm. Preferably, the height is between 120 and 140 mm, and most preferably it is about 130 mm.

The peripheral seal may comprise any one or more of: a weld; mechanical seal; heat seal; pressure seal; adhesive; solvent bond; ultraviolet bond; ultrasonic weld; laser weld; impulse weld; or friction weld. The peripheral seal may be re-sealable, for example through a resealable adhesive, an additional adhesive that is activated prior or during resealing, or a hook and loop/hook and hook fastener. Thus the peripheral seal can be easily constructed to suit the specific needs of the pouch.

One edge of the pouch may comprise a folded edge. A folded edge may comprise a fold between the two walls. All edges other than the folded edge may be formed by the peripheral seal. The pouch may be formed by folding the two walls to form the folded edge. Thus, the pouch can be flexibly constructed from folding material if required.

The pouch may comprise an interaction region defining a first point of contact for a user of the packaged catheter assembly. The interaction region may facilitate user access to the contents of the pouch. Actuation of the interaction region may create an opening in or near an upper edge of the peripheral seal. The catheter may be withdrawn through the opening. The catheter may be withdrawn through the opening. Thus, access to the pouch is provided at or near an upper edge of the pouch and as such, the fluid in the pool adjacent the base of the pouch is retained in the pouch while the catheter is withdrawn reducing the risk of spilling fluid outside the pouch.

The interaction region may comprise a breakable region of the peripheral seal. The breakable region may comprise a tear-away region which may comprise a tear line defining a line along which the pouch may be torn to at least partially separate the tear-away region from the remainder of the pouch, thereby creating an opening in the peripheral seal. The fluid collection bag may be arranged adjacent to the tear line. The tear-away region may comprise a tear start. The tear start may be provided at a first end of the tear-away region. The tear start may comprise a notch, or area of weakness, in one or both of the walls of the pouch. The tear start may define the first part of the pouch to be torn. The tear-away region may comprise a tear stop. The tear stop may be provided at a second end of the tear-away region. The tear stop may act to prevent or reduce the likelihood of the tear-away region being completely separated from the remainder of the packaging. A tear line may be defined between the tear start and the tear stop. The tear line may be configured to preferentially tear. The tear line may comprise a weakened region of the pouch. The tear line may be formed by any suitable means, such as laser scoring. Thus, the breakable region provides a convenient and easy means to open the pouch with minimal effort and no additional equipment/tools.

The tear-away region may be substantially triangular in shape having a sloped upper edge. The tear-away region may be tapered. The tear-away region may extend upwards more on one side than the other, preferably the tear-away region extends upwards more on the side corresponding to the tear start. The tear-away region may extend upwards no more than 200%, 100%, 75%, or 50% more on one side than the other. The tear-away region may extend upwards at least 25%, 50%, 75%, 100%, or 150% more on one side than the other. Preferably, the tear-away region extends upwards 50% more on one side than the other. Thus, the tear-away region is conveniently shaped to guide the user to grasp it on one side to open the pouch and also provides sufficient material for the user to securely grasp it on that side.

The interaction region may comprise an aperture. The aperture may be sized to allow a finger to pass through and grip the interaction region. Preferably, the aperture is located in the tear-away region. The aperture may be located on the same side of the pouch as the tear start. The aperture may be located adjacent to the tear start. The aperture may be any suitable shape, for example, circular, elliptical, square or irregularly shaped. Preferably, the aperture is circular. Thus, the user can more easily grasp the tear-away region and open the pouch.

The pouch may be formed from an opaque material. The pouch may have a textured or printed fabric-like appearance and/or feel. Where the pouch is opaque, the pouch may comprise a wetting marker to assist the user in locating the fluid reservoir within the pouch. Thus, the pouch is more comfortable and discreet for the user to carry in day-to-day life and the user can easily find and activate the fluid reservoir.

An axis parallel to the longest dimension of the stowed fluid collection bag may be arranged parallel to the tear line of the pouch and/or the opening formed in the pouch. This helps to protect the contents of the pouch from dirt when opening the pouch as the fluid collection bag provides an additional fluid impermeable barrier.

A first end of the stowed fluid collection bag (for example the inlet) may be arranged adjacent to one lateral edge of the pouch, or a first end of the opening. A second end of the stowed fluid collection bag (distal the first end) may be arranged adjacent a second end of the opening. For example, the first end of the stowed fluid collection bag may be arranged adjacent to the tear start. Thus, the fluid collection bag is positioned to ensure the distal end of the catheter is arranged along the same side of the pouch as the tear start. This helps the catheter to be withdrawn in a smooth and straight pulling action parallel to the lateral edge of the bag which reduces the force required to remove the catheter from the pouch.

The fluid collection bag may be configured to be withdrawn through the opening before the catheter. The fluid collection bag may be configured to withdraw the catheter from the pouch. Thus, the fluid collection bag facilitates removal of the catheter from the pouch without the user having to touch the catheter.

The fluid collection bag may be arranged to span across a majority of the width of the pouch. The fluid collection bag may span at least 50%, 60%, 70%, 80%, or 90% of the width of the pouch. Thus, the fluid collection bag forms a barrier across the opening in the pouch and protects the contents of the pouch.

The fluid collection bag may be arranged closer to the upper edge of the pouch than the base. The fluid collection bag may be arranged at least ½, ⅔, ¾ or ⅘ of the way from the base to the upper edge of the pouch. Thus, the fluid collection bag is arranged in the upper region of the pouch and close to the opening.

The fluid collection bag may obscure the catheter from the opening. The fluid collection bag may be arranged to span across a majority of the opening. The fluid collection bag may span at least 50%, 60%, 70%, 80%, or 90% of the opening. The fluid collection bag may obscure the rest of the contents of the bag from the opening. Thus, the user is less likely to see and then immediately grasp or touch the catheter itself.

The fluid collection bag may be configured to separate the two walls of the pouch. The fluid collection bag may be configured to separate the two walls of the pouch at the opening of the pouch. Thus, once the opening is formed in the pouch, the fluid collection bag removes the need for the user to separate the walls of the pouch which can be inconvenient or difficult, especially for users with reduced mobility.

The inlet of the fluid collection bag may be arranged adjacent to one lateral edge of the pouch and/or the opening. Where the packaged catheter assembly comprises a tear start, the inlet of the fluid collection bag may be arranged on the same side of the pouch as the tear start. Thus, the inlet is positioned to ensure the distal end of the catheter is arranged along the same side of the pouch as the tear start. This helps the catheter to be withdrawn in a smooth and straight pulling action parallel to the lateral edge of the bag which reduces the force required to remove the catheter from the pouch.

The fluid reservoir may be arranged within the pouch. The fluid reservoir may be arranged with its length parallel to one edge of the pouch. Preferably, the length of the fluid reservoir is parallel to the left and/or right lateral edge of the pouch. The fluid reservoir may be arranged adjacent to one edge of the pouch. Preferably, the fluid reservoir is arranged adjacent to the same edge of the pouch that the fluid reservoir's length is parallel to. Thus, the fluid reservoir is conveniently located against an edge of the pouch where it does not take up too much space.

The catheter may be arranged within the pouch in a shape having two opposing turns of at least 180 degrees along the catheter's length.

The catheter assemblies of each of the first to the eighth aspects may include any one or more features of a catheter assembly as defined in general terms, or according to the first to eighth aspects set out above. The catheter assemblies of the first to eighth aspects may comprise any of the optional features of the first to eighth aspects without necessarily including all the features required of them. That is to say, an optional feature which happens to be set out following one particular aspect does not necessarily apply only to that aspect, so, for example, the disclosure provides for the catheter assembly of the first aspect comprising a catheter having a proximal end for insertion into the body and a distal end; and a fluid collection bag arranged to receive fluid from the distal end of the catheter, wherein the fluid collection bag comprises a hand strap, but not necessarily comprising an outlet valve as described in relation to the third aspect.

According to a ninth aspect of the present invention, there is provided a method of manufacturing an intermittent catheter assembly comprising the steps of providing a catheter having a proximal end for insertion into the body and a distal end; and a fluid collection bag, arranging the fluid collection bag to receive fluid from the distal end of the catheter, wherein the fluid collection bag comprises a hand strap.

The method of the ninth aspect of the invention may be a method of manufacturing the catheter assembly of the first aspect of the invention, which, of course, may include any optional feature outlined above.

According to a tenth aspect of the present invention, there is provided a method of manufacturing a catheter assembly comprising the steps of providing a catheter having a proximal end for insertion into the body and a distal end, and a fluid collection bag, arranging the fluid collection bag to receive fluid from the distal end of the catheter, wherein the fluid collection bag comprises an outlet valve configured to control release of fluid out of the fluid collection bag in use, the outlet valve comprising: a valve base attached to a panel of the fluid collection bag, the valve base comprising a base opening for receiving a liquid output from the fluid collection bag; and an outlet element configured to be laterally rotatable relative to the valve base from a closed configuration to an open configuration.

The method of the tenth aspect of the invention may be a method of manufacturing the catheter assembly of the third aspect of the invention, which, of course, may include any optional feature outlined above.

According to a eleventh aspect of the present invention there is provided a method of forming a catheter assembly comprising the steps of providing a catheter having a proximal end for insertion into the body and a distal end, arranging a fluid collection bag to receive fluid from the distal end of the catheter, enclosing the catheter along the length of the catheter from the proximal end to the distal end with a sleeve, and arranging a fluid reservoir at the proximal end of the catheter, wherein the fluid reservoir is configured to release wetting fluid into the sleeve to wet the catheter and wherein the sleeve is in fluid communication with the fluid collection bag.

The method of the eleventh aspect of the invention may be a method of forming the catheter assembly of the fifth aspect of the invention, which, of course, may include any optional feature outlined above.

According to a twelfth aspect of the present invention, there is provided a method of forming a packaged catheter assembly comprising the steps of providing a fluid reservoir, a catheter, and a pouch, and arranging the fluid reservoir and catheter within the pouch; wherein the catheter comprises a proximal end for insertion into the body, a distal end, and a sleeve extending from the proximal end to the distal end, wherein an end of the sleeve comprises a reservoir adapter configured to: engage the fluid reservoir; receive a wetting fluid from the fluid reservoir into the sleeve to wet the catheter; and detach from the fluid reservoir to permit the catheter to pass through the reservoir adapter.

The method of the twelfth aspect of the invention may be a method of forming the packaged catheter assembly of the sixth aspect of the invention, which, of course, may include any optional feature outlined above.

The method may comprise arranging the reservoir adapter at the end of the sleeve. The method may comprise inserting the fluid reservoir into the reservoir adapter so as to block/inhibit the passage of the catheter therethrough. Thus, the catheter is protected by the fluid reservoir/reservoir adapter.

According to an thirteenth aspect of the present invention there is provided a method of forming a catheter assembly, the method comprising providing a fluid reservoir surrounded by a jacket and a catheter; wherein the catheter comprises a proximal end for insertion into the body, a distal end, and a sleeve extending from the proximal end to the distal end; wherein the jacket is configured to receive a wetting fluid from the reservoir and direct the wetting fluid into the sleeve at the proximal end to wet the catheter.

The method of the thirteenth aspect of the invention may be a method of forming the catheter assembly of the seventh aspect of the invention, which, of course, may include any optional feature outlined above.

The method may comprise forming the jacket around the fluid reservoir. The method may comprise arranging two strips of material, one on either side of the fluid reservoir. The method may comprise joining the strips of material around their edge to form the jacket with the fluid reservoir within it. The strips of material may be joined any one or more of: a weld; mechanical seal; heat seal; pressure seal; adhesive; solvent bond; ultraviolet bond; ultrasonic weld; laser weld; impulse weld; or friction weld. Thus, the jacket is conveniently formed around the fluid reservoir.

The method may comprise forming a wetting aperture in the jacket. The method may comprise providing a reservoir adapter at an end of the sleeve and inserting the jacket (containing the fluid reservoir) into the reservoir adapter.

According to a fourteenth aspect of the present invention there is provided a method of manufacturing a packaged catheter assembly, the method comprising the steps of providing a catheter, a pouch, and a fluid collection bag configured to receive fluid from the catheter in use, arranging the catheter and fluid collection bag within the pouch wherein the pouch comprises an interaction region to form an opening in the pouch and allow the fluid collection bag and catheter to be removed from the pouch, and the fluid collection bag is arranged adjacent to the opening.

The method of the fourteenth aspect of the invention may be a method of manufacturing the packaged catheter assembly of the eighth aspect of the invention, which, of course, may include any optional feature outlined above.

The method may comprise arranging the catheter in the pouch and then arranging the fluid collection bag in the pouch. This can ensure the fluid collection bag remains adjacent to the opening.

The method may comprise attaching the catheter to an inlet of the fluid collection bag. The method may comprise arranging the inlet on one side of the fluid collection bag.

According to an fifteenth aspect of the present invention there is provided a method of wetting a catheter having a proximal end for insertion into the body and a distal end, the method comprising the steps of releasing wetting fluid from a fluid reservoir at the proximal end of the catheter, into a sleeve that encloses the catheter, allowing the wetting fluid to flow through the sleeve from the proximal end to the distal end, and collecting excess wetting fluid in a fluid collection configured to receive fluid from a distal end of the catheter and the sleeve.

The method of the fifteenth aspect of the invention may be a method of wetting the catheter of the catheter assembly of the fifth aspect of the invention, which, of course, may include any optional feature outlined above and may be manufactured according to the eleventh aspect of the invention.

The method may comprise introducing the catheter by its proximal end into the urethra. The method may comprise allowing fluid to pass from the body and into the fluid collection bag via the catheter and/or the sleeve. Consequently, the method may be a method of using a catheter assembly.

According to a sixteenth aspect of the present invention, there is provided a method of unpackaging a packaged intermittent catheter assembly, the packaged catheter assembly comprising a catheter having a proximal end for insertion into the body and a distal end; and a fluid collection bag arranged to receive fluid from the distal end of the catheter, wherein the fluid collection bag comprises a hand strap, further wherein the catheter and fluid collection bag are arranged within a pouch.

The method of the sixteenth aspect of the invention may be a method of unpackaging a packaged catheter assembly of the second aspect of the invention, which, of course, may include any optional feature outlined above and may be manufactured according to the ninth aspect of the invention.

According to a seventeenth aspect of the present invention, there is provided a method of unpackaging a packaged catheter assembly, the packaged catheter assembly comprising a catheter assembly comprising the steps of providing a catheter having a proximal end for insertion into the body and a distal end, and a fluid collection bag, arranging the fluid collection bag to receive fluid from the distal end of the catheter, wherein the fluid collection bag comprises an outlet valve configured to control release of fluid out of the fluid collection bag in use, the outlet valve comprising: a valve base attached to a panel of the fluid collection bag, the valve base comprising a base opening for receiving a liquid output from the fluid collection bag; and an outlet element configured to be laterally rotatable relative to the valve base from a closed configuration to an open configuration, wherein the catheter and the fluid collection bag are arranged within a pouch.

The method of the seventeenth aspect of the invention may be a method of unpackaging a packaged catheter assembly of the fourth aspect of the invention, which, of course, may include any optional feature outlined above and may be manufactured according to the tenth aspect of the invention.

According to a eighteenth aspect of the present invention, there is provided a method of unpacking a packaged catheter assembly, the packaged catheter assembly comprising a fluid reservoir, a catheter, and a pouch containing the fluid reservoir and catheter, wherein the catheter comprises a proximal end for insertion into the body, a distal end, and a sleeve extending from the proximal end to the distal end, wherein an end of the sleeve comprises a reservoir adapter configured to engage the fluid reservoir, the method comprising the steps of releasing wetting fluid from the fluid reservoir into the sleeve, and detaching the fluid reservoir to permit the catheter to pass through the reservoir adapter.

The method of the eighteenth aspect of the invention may be a method of wetting a catheter from the packaged catheter assembly of the sixth aspect of the invention, which, of course, may include any optional feature outlined above and may be manufactured according to the twelfth aspect of the invention.

The method may comprise forming an opening in the pouch. The step of releasing wetting fluid may comprise rupturing the fluid reservoir, for example by pressing the activation or wetting marker. The method may comprise allowing fluid to flow through the reservoir adapter. Thus, the fluid reservoir may be easily activated to release wetting fluid into the sleeve.

The fluid reservoir may be detached from the reservoir adapter after the release of wetting fluid from the fluid reservoir. Thus, the catheter is adequately wetted before the fluid reservoir is removed.

The method may comprise withdrawing the catheter from the sleeve. Only part of the catheter, preferably including the proximal end of the catheter, may be withdrawn from the sleeve. The method may comprise passing the catheter through the reservoir adapter. The method may comprise bunching the sleeve. The catheter may be withdrawn from the sleeve after it has been wetted. The catheter may be withdrawn from the sleeve after the fluid reservoir has been removed from the sleeve. Thus, the fluid reservoir protects the catheter before use, and the catheter is easily withdrawn through the reservoir adapter.

The method may comprise introducing the catheter by its proximal end into the urethra. The method may comprise allowing fluid to pass from the body and into the fluid collection bag via the catheter and/or the sleeve. Consequently, the method may be a method of using a catheter assembly.

According to an nineteenth aspect of the present invention there is provided a method of wetting a catheter of a catheter assembly, the catheter assembly comprising the catheter having a proximal end for insertion into the body, a distal end and a sleeve extending from the proximal end to the distal end, and a jacket surrounding a fluid reservoir, the method comprising releasing wetting fluid from the fluid reservoir into the jacket and directing wetting fluid from the jacket into the sleeve at the proximal end of the catheter.

The method of the nineteenth aspect of the invention may be a method of unpackaging a packaged catheter assembly of the seventh aspect of the invention, which, of course, may include any optional feature outlined above and may be manufactured according to the thirteenth aspect of the invention.

The method may comprise activating the fluid reservoir to release wetting fluid via an interaction with the jacket. The method may comprise removing the jacket and fluid reservoir from the sleeve.

The method may comprise introducing the catheter by its proximal end into the urethra. The method may comprise allowing fluid to pass out of the body via the catheter. Consequently, the method may be a method of using a packaged catheter assembly.

According to a twentieth aspect of the present invention there is provided a method of unpackaging a packaged catheter assembly, the packaged catheter assembly comprising a catheter, a fluid collection bag configured to receive fluid from the catheter in use, and a pouch containing the catheter and fluid collection bag, the method comprising using an interaction region to form an opening in the pouch to allow the catheter and fluid collection bag to be removed from the pouch, wherein the fluid collection bag is arranged adjacent to the opening.

The method of the twentieth aspect of the invention may be a method of unpackaging a packaged catheter assembly according to the eighth aspect of the invention and may, of course, include any optional feature outlined above and the packaged catheter assembly may be manufactured according to the ninth aspect of the invention.

The method may comprise withdrawing the fluid collection bag from the pouch before the catheter. The method may comprise withdrawing the catheter from the pouch using the fluid collection bag.

The method may comprise unfolding the fluid collection bag. The method may comprise handling the fluid collection bag via one or more projections and/or one or more finger holes disposed on the fluid collection bag.

The method may comprise measuring the fluid contained in the fluid collection bag via a fill level indicator disposed on the fluid collection bag.

The method may comprise introducing the catheter by its proximal end into the urethra. The method may comprise allowing fluid to pass out of the body via the catheter. Consequently, the method may be a method of using a packaged catheter assembly.

The methods of the ninth to twentieth aspects of the present invention may of course individually include any one or more of the features, optional or otherwise, of one another and further may include any one or more of the features, optional or otherwise, of the first to eighth aspects of the present invention.

In any aspect the catheter is preferably a urinary catheter; and/or is an intermittent catheter. Thus, the features of aspects of the present invention allow intermittent male urinary catheters to be adequately wetted prior to use which can be more difficult than for other types of catheters which are generally shorter.

In any aspect, the catheter assembly is preferably a closed catheter assembly, which is that the catheter assembly comprises a catheter coupled to a fluid collection bag, such that it is configured to collect and retain fluid released from the body through the catheter. The fluid collected/catheter assembly may then be disposed of by the user when is convenient. Of course, embodiments of the invention may also be open catheter assemblies in that fluid is not collected or retained by the catheter assembly. Likewise in any aspect the catheter is preferably a urinary catheter; and/or is an intermittent catheter. Thus, the features of aspects of the present invention allow intermittent male urinary catheters to be adequately wetted prior to use which can be more difficult than for other types of catheters which are generally shorter.

As used herein, ranges and amounts can be expressed as “about” a particular value or range. About also includes the exact amount. For example, “about 5 mm” means “about 5 mm” and also “5 mm.” Generally, the term “about” includes an amount that would be expected to be within experimental error. The term “about” includes values that are within 5% less to 5% greater of the value provided. For example, “about 30 mm” means “between 28.5 mm and 31.5 mm.

### DETAILED DESCRIPTION OF THE INVENTION

In order that the invention may be more clearly understood one or more embodiments thereof will now be described, by way of example only, with reference to the accompanying drawings, of which:

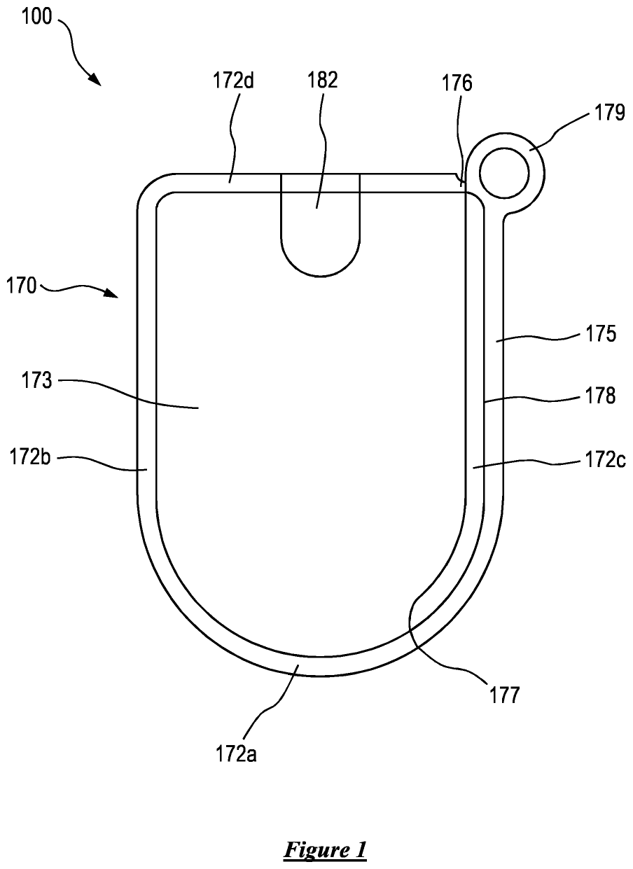

FIG. 1 is a front view of a first embodiment of a packaged catheter assembly;

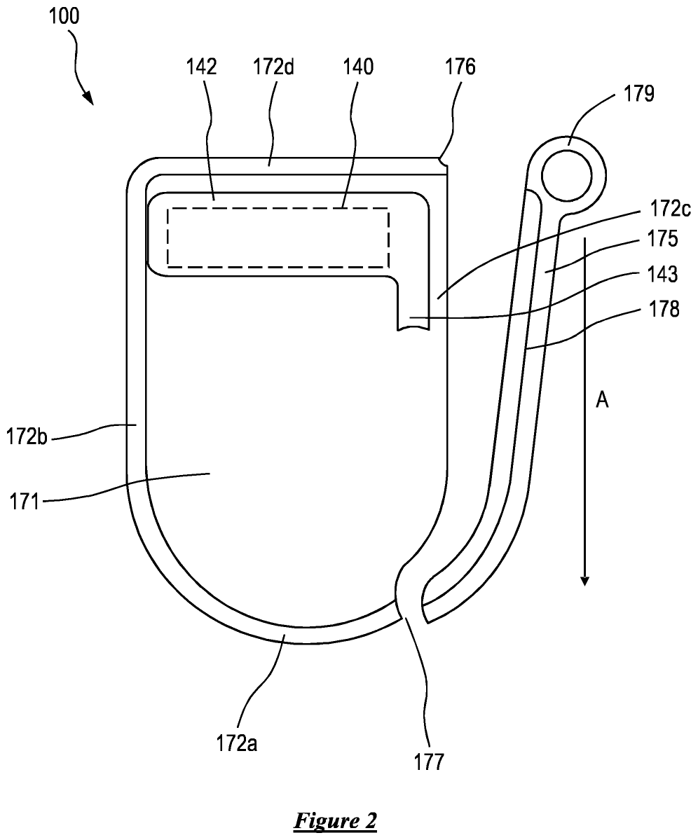

FIG. 2 is a cut-away front view of the packaged catheter assembly of FIG. 1 during forming an opening in the pouch;

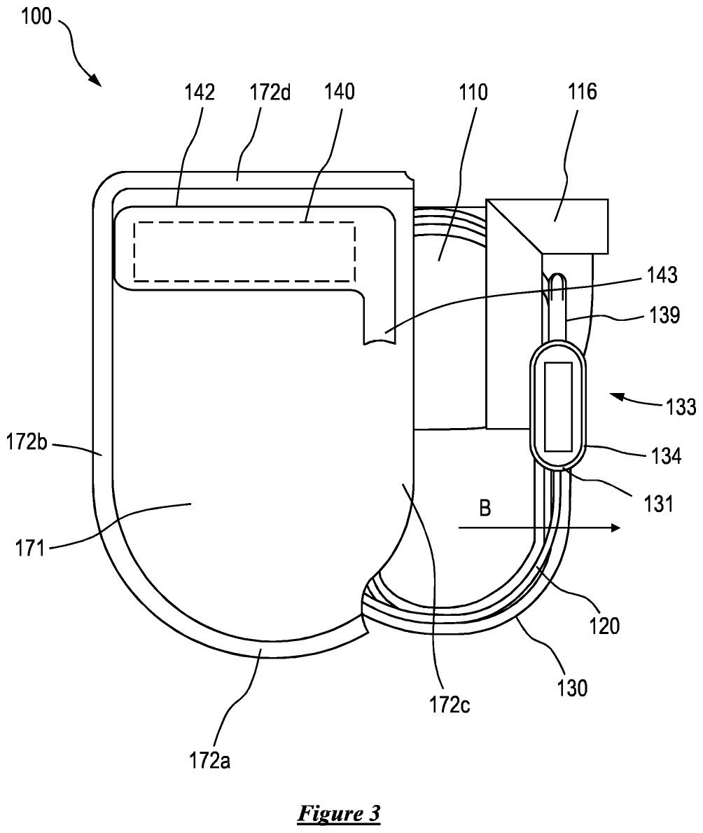

FIG. 3 is a cut-away front view of the packaged catheter assembly of FIG. 1 during withdrawal of the catheter assembly from the pouch;

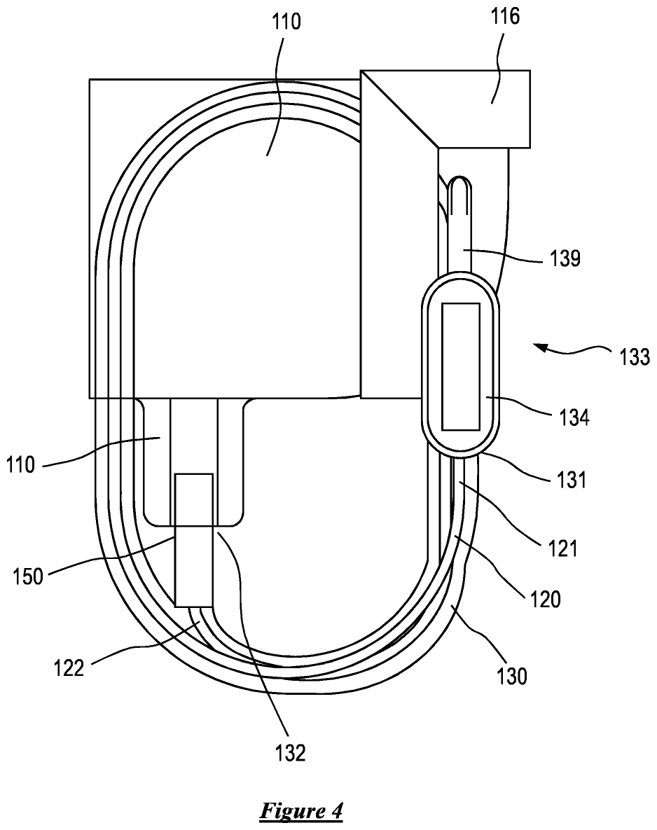

FIG. 4 is a front view of the catheter assembly of FIG. 1 where the catheter assembly has been removed from the pouch; and

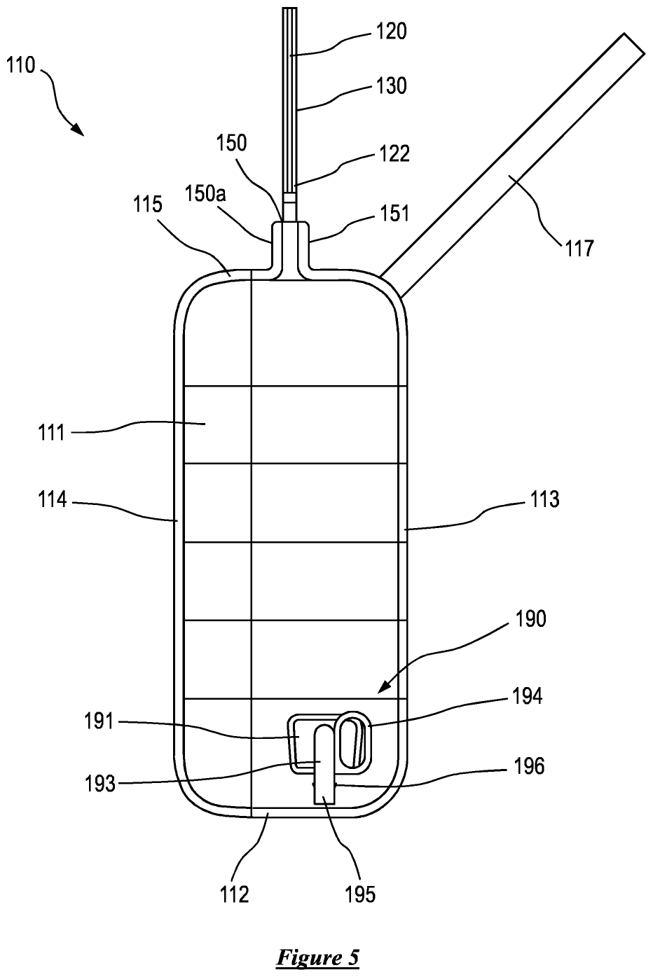

FIG. 5 is a front view of a fluid collection bag catheter assembly of FIG. 1;

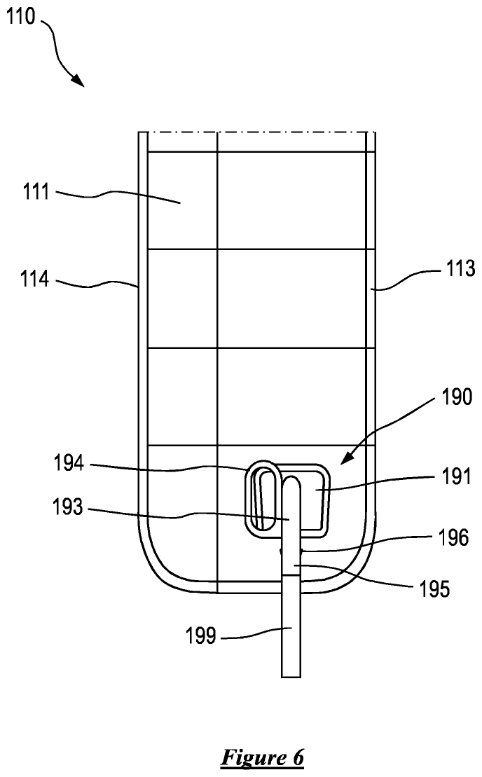

FIG. 6 is a partial front view of a fluid collection bag catheter assembly of FIG. 1;

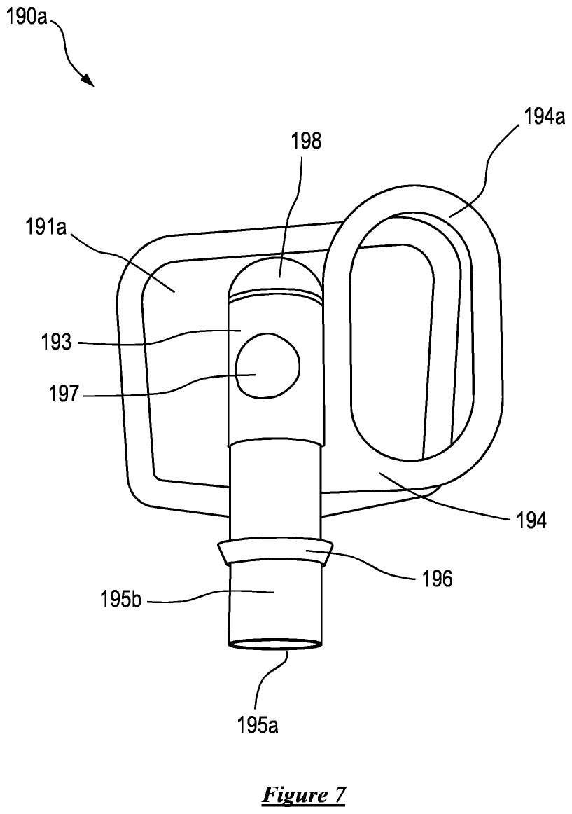

FIG. 7 is a front view of an outlet valve of the fluid collection bag of FIG. 6;

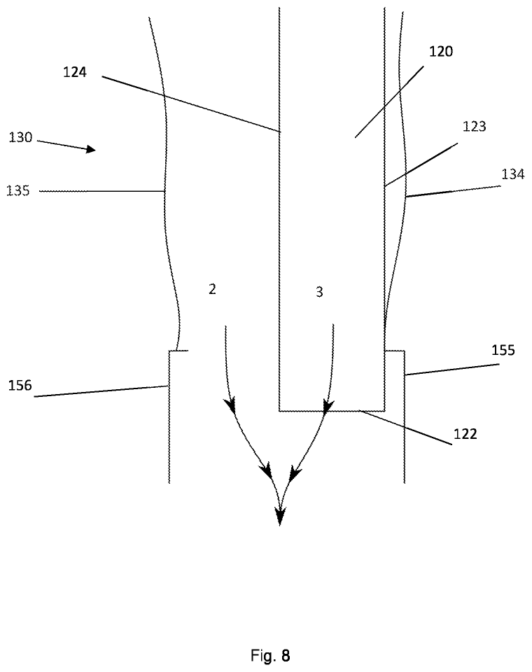

FIG. 8 is a cut-away view of a first example of the inlet of the catheter assembly of FIG. 1;

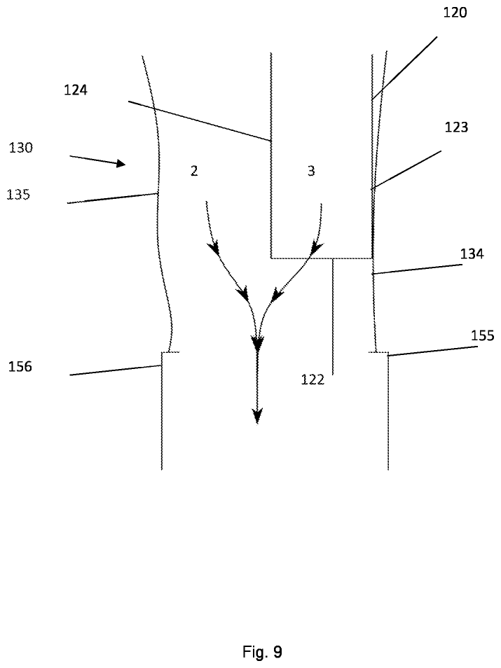

FIG. 9 is a cut-away view of a second example of the inlet of the catheter assembly of FIG. 1; and

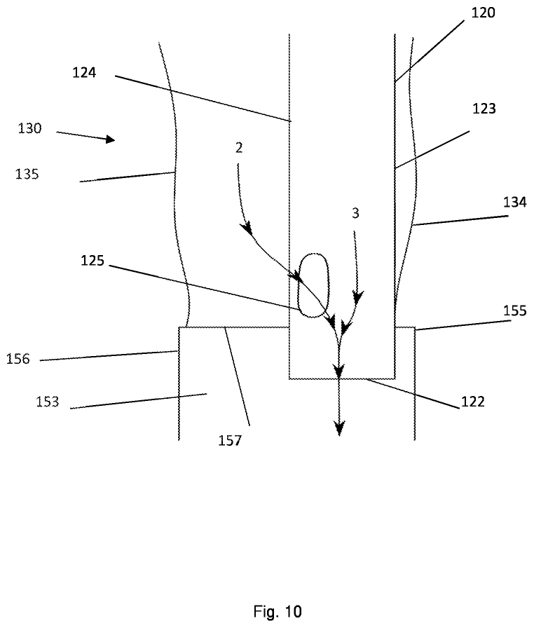

FIG. 10 is a cut-away view of a third example of the inlet of the catheter assembly of FIG. 1.

In the Figures, as is conventional, broken lines show hidden features.

Referring the Figures, an embodiment of a packaged catheter assembly 100 is shown.

The packaged catheter assembly 100 comprises a pouch 170 containing a fluid collection bag 110, a catheter tube, or “catheter” 120, a sleeve 130, and a fluid reservoir 140 comprising wetting fluid. The catheter 120 has a proximal end 121 for insertion into the body and a distal end 122, and the fluid collection bag 110 is attached to the distal end 122 of the catheter 120 and is arranged to receive fluid from the distal end 122 of the catheter 120, thereby forming a so-called “closed catheter assembly”. In this embodiment, the sleeve 130 and fluid reservoir 140 are also attached to the catheter 120 and so are also considered part of the “closed catheter assembly”.

In this embodiment, the catheter 120 is a male urinary catheter made from a hydrophilic thermoplastic elastomer (TPE). The sleeve 130 of this embodiment is a thermoplastic polyurethane (TPU) or low-density polyethylene (LDPE). Obviously those skilled in the art will be able to select suitable alternative materials.

The sleeve 130 of this embodiment is sized to enclose the catheter 120 along the majority of the length of the catheter 120, and preferably the entire length of the catheter 120. The sleeve 130 comprises a proximal end 131 corresponding to the proximal end 121 of the catheter 120 and a distal end 132 corresponding to the distal end 122 of the catheter 120. The distal end 132 of the sleeve 130 is attached to an inlet 150 of the fluid collection bag 110. The proximal end 131 of the sleeve comprises a reservoir adapter 133.

In this embodiment, the reservoir adapter 133 is generally tubular with a constant wall thickness and comprises a bore through its entire length. Of course, in other embodiments the wall thickness may vary allowing the size/shape of the reservoir adapter to be varied as desired. The reservoir adapter 133 comprises a distal end (not shown) with annular cross-section where it is attached to the proximal end 131 of the sleeve 130. The distal end comprises an internal diameter sized to allow the catheter 120 and wetting fluid to pass through the reservoir adapter 133 as described further below.

The reservoir adapter 133 is configured to releasably engage the fluid reservoir 140, and as such, a proximal end (not shown) of the reservoir adapter 133 distal from the sleeve 130 is configured to receive and retain the fluid reservoir 140. In this embodiment, the reservoir adapter 133 has an internal size and shape that matches the fluid reservoir 140 to allow the fluid reservoir to be push-fit into the reservoir adapter 133. This also allows the fluid reservoir 140 to be easily detached from the reservoir adapter 133 by pulling them apart. Of course, in other embodiments the fluid reservoir 140 may be received in a different way, such as through a bayonet or screw type fit, or through use of a temporary adhesive.

In this embodiment, the fluid reservoir 140 is an elongate rectangular burstable sachet of water. The fluid reservoir 140 is arranged with a first end engaged into the reservoir adapter 133. The reservoir adapter 133 extends around the first end of the fluid reservoir 140 and along the fluid reservoir 140 between 60-20% of the length of the fluid reservoir 140, for example 33%. Consequently, the proximal end of the reservoir adapter 133 comprises a generally elliptical annular cross section to allow an interference fit between the fluid reservoir 140 and reservoir adapter 133.

In this embodiment, the reservoir adapter 133 also comprises a midsection (not shown) between the annular distal end and larger elliptical proximal end enclosing the fluid reservoir 140. The midsection is tapered and has a convex shape to assist the user in gripping the reservoir adapter and using it as a handling element for the catheter 120 and/or sleeve 130. The midsection also acts as a funnel to deliver wetting fluid from the fluid reservoir 140 to the catheter 120/sleeve 130.

In this embodiment, the fluid reservoir 140 is be configured to retain 5 to 50 ml of wetting fluid, for example 12 ml. The fluid reservoir 140 has a height equal to between 20-50% the height of the pouch 170, for example 30%, and a width between 5-25% the width of the pouch 170, for example 15%. The height and width of the pouch 170 are defined as the distance between a base 172a and an upper edge 172d of the pouch, and a left lateral edge 172b and right lateral edge 172c of the pouch 170 respectively and as described in more detail below.

The fluid reservoir 140 also comprises a wetting marker (not shown) configured to allow the user to identify where to activate the fluid reservoir 140 to release wetting fluid. In this embodiment, the wetting marker comprises a logo/region of different colour or contrast from the rest of the fluid reservoir 140. In this embodiment, the wetting marker is positioned in a region of the fluid reservoir 140 that is not enclosed by the reservoir adapter 133, this allows the user to easily identify the wetting marker and activate the fluid reservoir 140. In other embodiments, the wetting marker may provide any suitable visual or tactile indication to assist the user in identifying and activating the fluid reservoir 140 to release wetting fluid.

In this embodiment, the fluid reservoir 140 is configured to release wetting fluid by rupturing upon depression/compression of the fluid reservoir 140, especially at a position corresponding to the wetting marker 144. The fluid reservoir 140 is configured to rupture and create a tear in the fluid reservoir 140 through which wetting fluid is released. In this embodiment, the fluid reservoir 140 is configured to create a tear in locations that are enclosed by the reservoir adapter 133. Thus, the reservoir adapter 133 is therefore configured to receive wetting fluid from the fluid reservoir 140 into the sleeve 130 to wet the catheter 120. To ensure that wetting fluid released from the fluid reservoir 140 is directed into the sleeve 130, the reservoir adapter 133 and/or fluid reservoir 140 may comprise a sealing element to prevent wetting fluid from escaping into the pouch 170, for example an O-ring provided around the end of the reservoir adapter 133 that engages the fluid reservoir 140. Such an O-ring may also facilitate the push-fit engagement of the fluid reservoir 140 and reservoir adapter 133.

In other embodiments, many other types of fluid reservoir would be suitable including different shapes and sizes of reservoir, or non-burstable reservoirs and other external sources of fluid. In this embodiment, the wetting fluid is water and interacts with the hydrophilic surface of the catheter 120 to render it lubricious. In other embodiments, other wetting fluids may be used and they may be polar (e.g. water-based) or non-polar (e.g. oil-based) depending on the catheter's surface properties.

In this embodiment, the fluid reservoir 140 comprises a thermoplastic polyurethane (TPU) or low-density polyethylene (LDPE). In this embodiment, the reservoir adapter 133 comprises a relatively rigid material compared to the sleeve 130, fluid reservoir 140 and catheter 120, for example high-density polyethylene (HDPE), and thus provides a handling element to facilitate movement of the proximal end 131 of the sleeve 130 up and down the catheter 120.

The pouch 170 is formed from a front wall 173 and rear wall 171 of identical shape and size, and a peripheral seal joining the periphery of the walls to form the pouch 170 and containing its contents (i.e. the closed catheter assembly).

The peripheral seal defines a base 172a, a left lateral edge 172b, a right lateral edge 172c and an upper edge 172d of the pouch 170. The left lateral edge 172b and right lateral edge 172c being defined as the left and right sides of the pouch 170 when viewing the pouch 170 with the rear wall 171 behind the front wall, the base 172a at the bottom of the pouch 170 and the upper edge 172d at the top of the pouch 170. The upper edge 172d is linear and the base 172a is outwardly curved, away from the upper edge 172d, and forms a curved bottom of the pouch 170. The peripheral seal thus defines a pouch 170 that is generally rectangular with a curved bottom and suitably has a width between the lateral edges of between 80-160 mm, preferably 100-140 mm, for example about 120 mm, and a height from the lowermost part of the base 172a to the upper edge 172d of between 100 to 250 mm, preferably 120 to 180 mm, more preferably 140 to 160 mm, for example about 150 mm. The base 172a defines the bottom of the pouch 170 in use, and the upper edge 172d the top. However, in other embodiments alternative shapes and sizes could be conceived, such as rectangular, elliptical or circular walls forming correspondingly shaped pouches, or even a tubular pouch or pouch with more than two walls, e.g. a prism or other polygon.

The peripheral seal may be formed from any suitable bonding/sealing methods such as chemical adhesives, chemical bonding, ultrasonic bonding, or mechanical bonding. In alternative embodiments, the pouch 170 may be formed from a single piece of material. The pouch 170 may then be formed by folding this single piece of material along a pouch fold line to form the walls. In addition, in some embodiments any number of folds may be used to form the pouch 170. For example, none of the edges may comprise a fold, one edge may comprise a fold, or two or more of the edges may comprise a fold.

In this embodiment, the pouch 170 is formed from an opaque material and as such provides a discrete package for its contents which can make the user more comfortable in carrying the packaged catheter assembly 100 in day-to-day life. In addition, the exterior of the pouch 170 may have a textured or printed appearance, for example a matte appearance, and feel to reduce the impression that it is a medical device. The front and/or rear wall of the pouch may comprise a wetting marker 182 which provides a visual/tactile guide to help the user locate the fluid reservoir 140 within the closed pouch and activate it to release wetting fluid. In this embodiment, the wetting marker 182 is located on the front wall 173 at the centre of the upper edge 172d of the pouch 170.

In this embodiment, the pouch 170 also comprises an interaction region. The interaction region forms the right side of the pouch 170 and spans between the upper edge 172d and base 172a. The interaction region is used to provide access to the pouch through/near to the right lateral edge 172c. In this embodiment, the interaction region comprises a tear-away region 175. The tear away region 175 is of equal width throughout its length. The tear-away region 175 comprises a tear start 176 at the point the upper edge 172d meets the tear-away region 175, a tear stop 177 at a corresponding point on the base 172a, and a tear line 178 spanning between the tear start 176 and tear stop 177. The tear line 178 comprises a line of weakness in the walls of the pouch 170, which may be formed by any suitable means but in this embodiment is laser etched. The tear start 176 comprises a notch to provide an area of weakness in the peripheral seal. This allows the tear-away region 175 to be separated from the pouch 170 by tearing the walls apart from the tear start 176 to the tear stop 177 along the tear line 178. Thus the tear is provided beneath the location of the right lateral edge 172c and thus the pouch 170 may be opened provided access to its contents. In other embodiments, the tear stop 177 may comprise a small aperture in the walls to prevent further tearing of the pouch 170.

The tear-away region 175 comprises a circular aperture 179 sized to allow a finger to pass through and grip the tear-away region 175. The circular aperture 179 is positioned in a corner where the upper edge 172d and right lateral edge 172c meet. To ensure the pouch 170 is fully sealed, the front wall 173 and rear wall 171 are sealed to one another over the entire tear-away region 175. In other embodiments, other seals such as a zip-lock arrangement, or hook and hook, hook and loop (VELCRO®) or the like may be used to provide access to the pouch (or to reseal the pouch after opening), and of course, the sizes and shapes set out above are exemplary.

In this embodiment, the catheter 120 is arranged within the pouch 170 in a curved, generally elliptical, curled/coiled configuration. The distal end 122 of the catheter 120 is arranged adjacent to the inner left lateral edge 172b near the base 172a of the pouch 170. The catheter 120 extends from a position where the proximal end 121 of the catheter 120 is adjacent the right lateral edge 172c down the inside of the pouch 170 into a first turn adjacent the base 172a of the pouch and then extends back up the pouch 170 adjacent to the left lateral edge 172b towards the upper edge 172d, into a second turn adjacent the upper edge 172d and then extends back down the pouch adjacent to the right lateral edge 172c towards the base 172a. The catheter 120 then comprises a third turn adjacent the base 172a where the distal end 122 of the catheter 120 connects to the fluid collection bag 110.

In this embodiment, the generally elliptical shape of the catheter 120 helps to maintain the contents of the pouch 170 in a stable position before use and also protects the catheter 120 from damage, for example kinking, due to mechanical stresses imposed on the pouch 170. Of course, the catheter 120 straightens as it is pulled out of the pouch, as described below.

In this embodiment, the fluid collection bag 110 is provided in a stowed configuration, which in this embodiment is a folded configuration, within the pouch 170 and is retained in the folded configuration by the walls of the pouch 170 which provide compressive pressure to the folded fluid collection bag 110. In other embodiments, the fluid collection bag 110 may be retained in the folded configuration by any other suitable means, such as an elastic band, paper band or closure. The folded fluid collection bag 110 is generally rectangular in shape and is arranged within the pouch 170 just below the upper edge 172d and with an axis of the rectangular folded fluid collection bag 110 approximately parallel to the upper edge 172d.

The fluid collection bag 110 comprises an inlet 150 to receive the distal end 122 of the catheter 120 adjacent to the left lateral edge 172b of the pouch 170, and the opposite end of the bag 110 is positioned adjacent to the right lateral edge 172c of the pouch 170. The fluid collection bag 110 is therefore arranged adjacent to the opening formed in the pouch 170 by the tear-away region 175 as described further below and spans between the left lateral edge 172b and right lateral edge 172c.

In this embodiment, the sleeve 130 is arranged around the catheter 120 and covering the majority of the length of the catheter 120. The distal end 132 of the sleeve 130 is attached to the inlet 150 of the fluid collection bag 110, and as described above, the proximal end 131 of the sleeve 130 is attached to the reservoir adapter 133. The reservoir adapter 133 is positioned at the proximal end 121 of the catheter 120 and adjacent the right lateral edge 172c of the pouch 170. The fluid reservoir 140 extends adjacent the inner upper edge 172d from the inner left lateral edge 172b to the inner right lateral edge 172c of the pouch 170. The fluid reservoir of this embodiment is attached to a wall of the pouch, e.g. by adhesive, to prevent it being accidentally removed from the pouch. The fluid reservoir 140 can therefore be activated by pressing the walls of the sealed closed pouch 170 together adjacent the upper edge 172d. Of course, in other embodiments, the catheter 120, fluid collection bag, sleeve 130 with reservoir adapter 133 and fluid reservoir 140 may be arranged differently within the pouch 170.

In this embodiment, the pouch 170 comprises a reservoir jacket 142 configured to enclose the fluid reservoir 140. The reservoir jacket 142 comprises an impermeable material, for example, silicon, a thermoplastic polyurethane (TPU) or low-density polyethylene (LDPE). The reservoir jacket 142 is rectangular and spans across the width of the pouch 170 adjacent to the upper edge of the pouch 170. The reservoir jacket 142 further comprises a wetting aperture 143 arranged at a position adjacent to the opening formed in the pouch 170 by actuation of the interaction region. The wetting aperture is configured to receive an inserter tip 139 of the reservoir adapter 133 and to direct wetting fluid from the reservoir jacket 142 and/or fluid reservoir 140 into the sleeve 130 and onto the catheter 120. In this embodiment, the wetting aperture is resiliently deformed by insertion of the inserter tip 139 therein. Consequently, the wetting aperture provides a fluid tight seal around the inserter tip 139. In other embodiments, the wetting aperture may fit to the reservoir adapter 133 through a different type of fitting, such as a screw or bayonet fitting. This ensures that wetting fluid released by the fluid reservoir 140 is directed into the sleeve 130. Of course, in other embodiments, the reservoir jacket 142 may have a different shape and size and the wetting aperture may be in a different position. Alternatively, the reservoir jacket 142 may couple to the reservoir adapter 133 via a different method, such as being inserted into the reservoir adapter 133.

Furthermore, as the reservoir jacket 142 comprises the wetting aperture, pressure applied to the fluid reservoir 140 to rupture it does not also rupture the reservoir jacket 142, as any increase in pressure within the reservoir jacket 142 is relieved through the wetting aperture 143. Thus, the reservoir jacket 142 remains impermeable to wetting fluid and can direct it into the sleeve 130 irrespective of the location in which the fluid reservoir 140 ruptures.

In this embodiment, the reservoir jacket 142 is attached to the inside of the pouch 170, for example by welding or gluing of the reservoir jacket 142 to the pouch 170. This ensures that the reservoir jacket 142 and fluid reservoir 140 are not removed from the pouch 170 unnecessarily or accidentally, which makes the packaged catheter assembly 100 more convenient to use.

Referring to FIGS. 5 to 7, the fluid collection bag 110 comprises a front panel 111, a rear panel of identical shape and size (not shown), and a peripheral bond joining the periphery of the panels to form the bag 110. The peripheral bond defines a base 112, a right lateral edge 113, a left lateral edge 114 and an upper edge 115. The right lateral edge 113 and left lateral edge 114 being defined as the right and left sides of the bag 110 when viewing the bag 110 with the rear panel behind the front panel 111, the base 112 at the bottom of the bag 110 and the upper edge 115 at the top of the bag 110. The peripheral bond thus defines a bag 110 that, when unfolded, is generally rectangular and suitably has a width between the lateral edges of at least 10, 12, 15 or 20 cm. The bag may have a width no more than 25, 20, 15, 12 or 10 cm. Preferably, the width is between 10 and 20 cm, most preferably between 10 and 15 cm, for example 12 cm. A height from the base 112 to the upper edge 115 may be at least 25, 30, 35 or 40 cm. The height of the bag may be no more than 45, 40, 35 or 30 cm. The height may be between 25 and 40 cm, 28 and 34 cm, or most preferably 29 and 31 cm, for example 30 cm. The bag may be configured to hold at least 500, 700, or 1000 ml of fluid, and may hold no more than 1000 ml. For example, it may hold at least 700 ml of fluid. The base 112 defines the bottom of the bag 110 in use, and the upper edge 115 the top. However, in other embodiments alternative shapes and sizes could be conceived, such as elliptical or circular panels forming correspondingly shaped bags, or even a tubular bag or bag with more than two panels, e.g. a prism or other polygon.

The front panel 111 and rear panel are both substantially formed from an opaque material. In this embodiment, 70% of the fluid collection bag 110 is formed from an opaque material. The remaining 30% of the fluid collection bag 110 is located proximal to the left lateral edge 114 and spans between the upper edge 115 and the base 112.

In this embodiment, the panels comprise a flexible plastics material, for example polypropylene (PP), polyethylene terephthalate (PET), low density polyethylene (LDPE), metalized polyester (MET PET), orientated polypropylene (OPP) or polyvinyl chloride (PVC).

In this embodiment, the inlet 150 is positioned at the upper edge 115 the peripheral bond. The inlet 150 is located at the end of a spout 150a. The spout 150a extends from the upper edge 115 of the fluid collection bag 110 equidistant between the corner where the upper edge 115 and left latera ledge 114 meet and the corner where the upper edge 115 and right lateral edge 113 meet. The spout 150a has a length, i.e. the distance from where the spout 150a joins the upper edge 115 to the inlet 150, of about 8 cm.

In this embodiment, the fluid collection bag 110 comprises a hand strap 117. The hand strap 117 is arranged adjacent to the inlet 150 on a corner of the fluid collection bag 110 where the upper edge 115 and right lateral edge 113 meet. The hand strap 117 is joined to the fluid collection bag 110 by a weld. Thus, the hand strap 117 can be joined to the fluid collection bag 110 simultaneously with the formation of the peripheral bond.

The hand strap 117 provides for a convenient and easy way of holding the fluid collection bag 110, where the hand strap 117 can be temporarily worn around the user's wrist or be easily held. Moreover, the hand strap 117 allows the fluid collection bag 110 to be easily manipulated by the user during use, meaning that the fluid collection bag 110 can be easily handled and manoeuvred by the user as is necessary. Further, the dimensions of the hand strap (as described below) means that the fluid collection bag 110 can be hanged hung from any suitable article, such as a wheelchair handle or bed post, when the catheter assembly is in use.

The hand strap 117 is arranged at the opposite end of the fluid collection bag 110 to an outlet valve 190a (described further below) configured to control release of fluid out of the fluid collection bag 110 in use. This facilitates the flow of fluid within the fluid collection bag 110, including the draining of liquid, toward the outlet valve 190a and away from the inlet 150, when in use.

The hand strap 117 is formed of an opaque material, such as polyethylene terephthalate, polypropylene or low-density polyethylene.

The hand strap 117 has a length (measured from the point at which it is joined to the bag 110 to its furthest point) of 15 cm and a width of 1 cm. The width of the hand strap 117 is constant along its entire length. In this embodiment, the length of the hand strap 117 is fixed, i.e. not adjustable.

The hand strap 117 is formed of a single, continuous loop of material which is joined at two ends to the fluid collection bag 110. The hand strap 117 is joined to the fluid collection bag 110 at two positions on the fluid collection bag 110. In the present embodiment, each of these positions are substantially the same and are each located on the corner where the upper edge 115 and right lateral edge 113 meet. (In this embodiment the distance from one end of the strap to the opposite end, is 30 cm, i.e. 2×15 cm as both ends are attached to the same point.)

In this embodiment, the catheter tube has a length of 35 cm and a width defined as CH14 by the French System, i.e. about 0.5 cm. The catheter is formed of polyolefin material comprising polyethylene and polypropylene.

Referring to FIGS. 5 to 7, the fluid collection bag 110 comprises an outlet valve 190a. The outlet valve 190a is configured to control release of fluid out of the fluid collection bag 110 in use.

The outlet valve 190a is arranged on the front panel 111 of the fluid collection bag 110. The outlet valve 190a is arranged proximal to the base 112, off-centre toward the right lateral edge 113.

The outlet valve 190a comprises a valve base 191a attached to the front panel 111 of the fluid collection bag 110 by a weld. In the present embodiment, the valve base 191a is trapezoidal. The valve base has a height of 8 cm and a width, at its widest part, of 10 cm. The valve base 191a is formed of a material more rigid than the material from which the fluid collection bag 110 is formed. In the present embodiment, the valve base 191a is formed of polypropylene.

The valve base 191a comprises a base opening (not shown) for receiving a liquid output from the fluid collection bag 110. The base opening is an aperture of any suitable size and shape but is preferably a circular aperture having an diameter of at least 0.5 cm.

The outlet valve 190a further comprises an outlet element 193 configured to be laterally rotatable relative to the valve base 191a from a closed configuration to an open configuration. The outlet element 193 is rotatable about a vertical axis of rotation when in use. This means that the fluid collection bag 110, comprising an upper edge, left lateral edge, right lateral edge and base, comprises a vertical axis parallel to the left and right lateral edges, and it is about this axis which the outlet element 193 is rotatable.

The outlet element 193 is housed in a housing 198 (best seen in FIG. 7) which is formed integrally with the valve base 191a and sized to receive the outlet element 193 within it, so as to be rotatable between the open and closed configurations.

The outlet element 193 is rotatable such that the outlet element 193 moves through an arc of rotation. At a first end of the arc of rotation, the outlet element 193 is in the closed configuration, and at a second end of the arc of rotation, the outlet element 193 is in the open configuration. Moreover, the outlet element 193 is rotatable about an axis parallel to the plane of the front panel 111 of the fluid collection bag 110 to which the valve base 191a is attached.

The outlet element 193 is has a hollow tubular form with an internal cavity/bore of approximately 1 cm and comprises a circular aperture 197 having a diameter of 1 cm. The aperture 197 opens into, and is hence in fluid communication with the internal cavity of the outlet element 193. The aperture 197 is positioned in an upper portion of the outlet element 193. When the outlet element 193 is in the closed configuration, the aperture 197 is positioned facing outwardly relative to the fluid collection bag 110, facing away from the valve base 191a. In this configuration, the aperture 197 is not aligned with the base opening. Moreover, in the closed configuration, the base opening is aligned with a solid portion of the outlet element 193, therefore, flow of liquid from the fluid collection bag 110 through the base opening is prevented.

When the outlet element 193 is in the open configuration, the aperture 197 is positioned facing inwardly relative to the fluid collection bag 110, facing toward the valve base 191a. In this configuration, the base opening and aperture 197 are in fluid communication. As shown in FIG. 6, liquid 199 within the fluid collection bag 110 can therefore drain from the bag 110 via the base opening, aperture 197 and downwardly through the outlet element and through a connector portion 195b (further described below) and then into a receptacle or toilet or similar.

The connector portion 195b is a hollow cylindrical bore extending from the outlet element such that fluid entering the outlet element 193 via the aperture 197 flows downwardly, through the outlet element 193 and into, through the connector portion 195b and out of a connector portion outlet 195a located at a distal end of the connector portion 195b. The connector portion outlet 195a has a diameter of 1 cm. The connector portion 195b and outlet element 193 each have an inner diameter of 1 cm.

The outlet element 193 comprises a handle 194 arranged between the housing 198 and the connector portion 195b. The handle 194 and connector portion 195b are integrally formed with the outlet element 193 and are formed as a single moulding. The handle 194 is arranged at approximately 90 degrees anti-clockwise relative to the position of the aperture 197. This means that when the outlet element 193 is in the closed position, the handle 194 is arranged against the trapezoidal section of the valve base 191a. Lateral rotation of the outlet element 193 using the handle 194 by 180 degrees moves the handle 194 from a first position closer toward the right lateral edge 113 of the fluid collection bag 110 to a second position closer toward the left lateral edge 114 of the fluid collection bag 110, i.e. to the open position. Simultaneously, this rotates the outlet element 180 degrees such that the aperture is rotated from a position where the aperture 197 is facing away from the valve base 191a and the aperture 197 is not aligned with the base opening, to a position where the aperture is facing toward the valve base 191a and the aperture 197 is in fluid communication with the valve base 191a.

The handle 194 is inhibited from rotating more than 180 degrees clockwise or anti-clockwise due to obstruction by the valve base 191a.

The handle 194 is planar such that when the outlet element 193 is in the open or closed configuration, the handle 194 lies flat against the valve base 191a and only minimally protrudes away from the fluid collection bag 110.

The handle 194 is operable to rotate the outlet element 193 between the closed configuration and the open configuration. The handle 194 is rotatable from a position closer to the right lateral side 113 to a position closer to the left lateral side 114. Such rotation moves the outlet element 193 from the closed configuration to the open configuration.

The handle 194 has a length of 8 cm and a width of 4 cm. The handle 194 is sized to allow a user to easily grip it between at least their thumb and index finger, in particular for users with limited dexterity. The handle 194 is elliptical in shape.

The handle 194 comprises a finger-hole 194a. The finger-hole 194a is sized to allow at least one of the user's fingers to pass through it to facilitate rotation of the handle 194.

The handle 194 extends above the outlet element 193 and valve base 191a to facilitate gripping by the user. When in the closed or open configuration, the handle extends laterally further than the valve base 191a.

The connector portion 195b is configured for use with tubing (not shown) having a diameter marginally smaller than that of the connector portion 195b, such that, on insertion of the tubing onto a distal end of the connector portion 195b, over the connector outlet 195a, in use, the tubing attaches fluid-tight to the connector portion 195b. The annular flange 196 arranged proximal to the connector outlet 195a engages with the tubing.

The valve base 191a, including the housing 198, and outlet element 193, including the connector portion 195b are each formed of a material which is more rigid than the relatively flexible material from which the fluid collection bag is formed. In the present embodiment, the valve base 191a, and outlet element 193 are each formed of polypropylene.

The outlet element 193 protrudes away from the valve base 191a by 1.5 cm. The flange 196 protrudes away from the housing 198 by 0.3 cm.

Each component of the outlet valve 190a is formed of opaque materials.

Referring to FIGS. 1 to 4, in this embodiment, the packaged catheter assembly 100 is provided with the folded fluid collection bag 110, catheter 120, sleeve 130 and fluid reservoir 140 all contained with the pouch 170 as mentioned above. To wet the catheter 120 so that it is ready for use, the user may release the wetting fluid from the fluid reservoir 140 into the sleeve 130. This may be done by any suitable means, but preferably by applying pressure to the outside of the pouch 170 at a region corresponding to the position of the fluid reservoir 140 by pressing the wetting marker 182.

As described above, in this embodiment, the fluid reservoir 140 is configured to rupture under external pressure and create a tear in the fluid reservoir 140 at a position that is enclosed by the reservoir adapter 133. As such, the wetting fluid is released into the reservoir adapter 133 where it is able to flow through the bore of the reservoir adapter 133 along the length of the catheter 120 between the catheter 120 and sleeve 130.

Referring to FIGS. 2 and 3, in this embodiment, the user may then access the catheter 120 by creating an opening in the pouch 170 using the interaction region. The aperture 179 is grasped in one hand, and the pouch 170 below the tear line 178 in another, and the tear-away region 175 is torn from the tear start 176 to the tear stop 177 along the tear line 178 in a direction corresponding to arrow A in FIG. 2. Thus, the pouch 170 is torn between the upper edge 172d and base 172a at a location below the right lateral edge 172c, as such, an opening is formed in the pouch 170 that may be used to access its contents. In this embodiment, the tear-away region 175 is fully separated from the pouch 170 to improve access to the contents of the pouch 170. In other embodiments, the tear-away region 175 may not be fully separated from the pouch 170 to reduce the number of separate parts and make the pouch 170 easier to handle. Other embodiments may also feature other ways to access the pouch for example a zip-lock seal.

In this embodiment, the closed catheter assembly (i.e. catheter 120 attached to the sleeve 130, reservoir adapter 133, fluid reservoir 140, and fluid collection bag 110) may then be removed from the pouch 170. The folded fluid collection bag 110 is easily accessible to the user as it is directly accessible via the opening in the pouch 170. To withdraw the closed catheter assembly from the pouch 170 the user pulls the closed catheter assembly laterally out of the pouch 170 in the direction of arrow B in FIG. 3.

In this embodiment, the fluid collection bag 110 may then be unfolded so that it is ready to accept fluid from the catheter 120. The catheter 120 may be handled via the sleeve 130 and/or the reservoir adapter 133.

In this embodiment, the fluid reservoir 140 must be removed from the reservoir adapter 133 to allow the proximal end of 121 of the catheter 120 to be withdrawn from the sleeve 130 and inserted into the body. The fluid reservoir 140 is removed from the reservoir adapter 133 by simply pulling the reservoir adapter 133 out of the pouch and hence away from the fluid reservoir 140 which is retained within the pouch. Now that the fluid reservoir 140 is removed from the reservoir adapter 133, the catheter 120 is able to be passed through the reservoir adapter 133 for use. In other embodiments, the fluid reservoir 140 may be actively disengaged by twisting or otherwise activating the reservoir adapter 133 to release the sleeve 130 for example.

The steps of releasing wetting fluid into the sleeve 130, unfolding the fluid collection bag 110, and disengaging the fluid reservoir 140 as described above may be performed in any order depending on the construction of the packaged catheter assembly and the preference of the user.

The catheter 120 is now ready for use. The sleeve 130 may be used to handle the catheter 120 and the reservoir adapter 133 may be used to progressively introduce the proximal end 121 of the catheter 120 into the urethra until fluid flows through the catheter 120 from the bladder. Fluid flowing through the catheter 120 is collected in the fluid collection bag 110. After use, in this embodiment, as the catheter 120 is withdrawn from the body, the reservoir adapter 133 may be used to cover the length of the catheter 120 with the sleeve 130 and the packaged catheter assembly 100 may then be disposed of.

As mentioned above, in this embodiment the catheter 120 and sleeve 130 are in fluid communication with the fluid collection bag 110 via the inlet 150. The configuration of the catheter 120, sleeve 130, inlet 150 and bag 110 is relatively flexible and many different configurations could be employed in embodiments of the invention.

Referring to FIG. 8, in a first example of the configuration for the catheter 120, sleeve 130 and inlet 150 fluid passes directly from the sleeve 130 to the inlet 150 and directly from the catheter 120 to the inlet 150. In this example, a right lateral side 123 of the catheter 120 is attached to a right side 155 of the inlet 150 and there is a gap between a left lateral side 124 of the catheter 120 and the respective left side 156 of the inlet 150. In addition, the distal end 122 of the catheter 120 is within the inlet 150, and the sleeve 130 is sealed around a periphery of the inlet 150. As such, fluid travelling within the sleeve 2 is directed into the inlet 150 directly via the gap between the left lateral side 124 of the catheter 120 and the left side 156 of the inlet 150. In addition, fluid travelling within the catheter 3 passes out the distal end 122 and into the inlet 150 directly. In other examples, the catheter 120 may be positioned differently and may be attached to the left side 156 of the inlet 150, or to a front or rear side.

Referring to FIG. 9, in a second example fluid passes from the catheter 120 into the sleeve 130 and then into the inlet 150. In this example, the right lateral side 123 of the catheter 120 is attached to a left side 134 of the sleeve 130 and a distal end 122 of the catheter 120 is not within the inlet 150. As in the first example, the sleeve 130 is sealed around a periphery of the inlet 150. As such, fluid travelling within the sleeve 2 is directed into the inlet 150 directly and fluid travelling within the catheter 3 passes out the distal end 122 into the sleeve 130 and then from there into the inlet 150. In other examples, the catheter 120 may be positioned differently and may be attached to a left side 135 of the sleeve 130, or indeed to a front or rear side. Furthermore, the distal end 122 of the catheter 120 may be blocked and an outlet (not shown) may be provided in a lateral side of the catheter 120 to allow fluid to pass out into the sleeve 130.

Referring to FIG. 10, in a third example fluid passes from the sleeve 130 into the catheter 120 and then into the inlet 150. The configuration of this example is similar to the first example shown in FIG. 12 and so only the differences are described. In this example, the inlet 150 is sealed between the left side 156 and right side 155 such that the catheter 120 is sealed on all sides. As such, fluid may only pass from the sleeve 130/catheter 120 into the inlet 150 if it is within the catheter 120. The left lateral side 124 of the catheter 120 comprises an oval sleeve intake opening 125 adjacent to the inlet 150 which permits the passage of fluid travelling within the sleeve 3 into the catheter to join fluid travelling within the catheter 2. From here, the fluid 2, 3 passes out the distal end 122 of the catheter 120 and into the inlet 150. Many other configurations may be possible to achieve the same effect, for example, the sleeve 130 may be attached to the outer sides of the catheter 120 itself.

Before use, providing the user does not want the fluid collection bag 110 to drain from the outlet valve 190a during urination, the user must ensure that the outlet valve 190a is in a closed configuration. In this configuration, the handle 194 is arranged closer to the right lateral edge 113 of the fluid collection bag 110 and the aperture 197 therefore positioned facing away from the valve base 191a such that the base opening is adjacent to a solid portion of the outlet element 193. After urination, when the user wishes to drain the fluid collection bag 110, the user grips the handle 194, optionally using the finger-hole 194a, and rotates the handle 194 approximately 180 degrees toward the left lateral edge 114 of the fluid collection bag 110. Simultaneously, the outlet element 193 is rotated. This brings into alignment the aperture 197 and the base opening. As such, fluid from the fluid collection bag 110 can drain through the base opening in the valve base 191a, through the aperture 197, into the outlet element 193 and downwardly through the connector portion 195b and out of the outlet 195a.

While the embodiments disclosed above is described as a closed catheter assembly in that fluid is collected in a fluid collection bag, other embodiments may not comprise a fluid collection bag and may be open catheter assemblies in that fluid is not collected during use.

The one or more embodiments are described above by way of example only. Many variations are possible without departing from the scope of protection afforded by the appended claims.

1. An intermittent catheter assembly comprising a catheter having a proximal end for insertion into the body and a distal end, a fluid collection bag arranged to receive fluid from the distal end of the catheter, wherein the fluid collection bag comprises an outlet valve configured to control release of fluid out of the fluid collection bag in use, the outlet valve comprising: a valve base attached to a panel of the fluid collection bag, the valve base comprising a base opening for receiving a liquid output from the fluid collection bag; and an outlet element configured to be laterally rotatable relative to the valve base from a closed configuration to an open configuration.

2. An intermittent catheter assembly according to claim 1 wherein the fluid collection bag comprises a base and an upper edge and first and second lateral edges.

3. An intermittent catheter assembly according to claim 1 or 2, wherein the outlet element comprises a handle, which is operable to rotate the outlet element between the closed configuration and the open configuration.

4. An intermittent catheter assembly according to claim 3 when dependent on claim 2 wherein the handle is rotatable from a position closer to the first lateral edge to a position closer to the second lateral edge.

5. An intermittent catheter assembly according to claim 3 or 4, wherein the handle is planar.

6. An intermittent catheter assembly according to any of claim 3 to 5, wherein the handle comprises a finger-hole.

7. An intermittent catheter assembly according to any of claim 3 to 6, wherein the handle extends above the outlet element.

8. An intermittent catheter assembly according to any of claim 3 to 7, wherein the handle extends above the valve base.

9. An intermittent catheter assembly according to claim 5, wherein the plane of handle is aligned with the plane of the panel of the fluid collection bag to which the valve base is attached, when in the closed configuration.

10. An intermittent catheter assembly according to claim 5 or 6, wherein the plane of handle is aligned with the plane of the panel of the fluid collection bag to which the valve base is attached, when in the open configuration.

11. An intermittent catheter assembly according to any of claims 3 to 10, wherein the handle rotates by at least 90, 120, 150 or 180 degrees between the closed configuration and the open configuration.

12. An intermittent catheter assembly according to any preceding claim, wherein the valve base comprises a housing which houses the outlet element and in which the outlet element rotates.

13. An intermittent catheter assembly according to any preceding claim, wherein the outlet element is rotatable about a vertical axis of rotation when in use.

14. An intermittent catheter assembly according to any preceding claim, wherein the outlet element is rotatable about an axis parallel to the longest dimension of the fluid collection bag.

15. An intermittent catheter assembly according to any preceding claim, wherein the outlet element is rotatable about an axis parallel to the plane of the panel of the fluid collection bag to which the valve base is attached, when in the closed configuration or the open configuration.

16. An intermittent catheter assembly according to any preceding claim, wherein the fluid collection bag comprises a front panel and a rear panel.

17. An intermittent catheter assembly according to claim 16, wherein the outlet valve is arranged off-centre when the fluid collection bag is viewed with the rear panel behind the front panel.

18. An intermittent catheter assembly according to claim 17, wherein the outlet valve is arranged off-centre toward a left lateral edge or toward a right lateral edge of the fluid collection bag.

19. An intermittent catheter assembly according to any preceding claim, wherein the outlet element is rotatable about an axis parallel to the plane of a front panel of the fluid collection bag.

20. An intermittent catheter assembly according to claim 19, wherein the outlet valve is arranged on the front panel of the fluid collection bag.

21. An intermittent catheter assembly according to any preceding claim, wherein the catheter is an intermittent catheter.

22. An intermittent catheter assembly according to any preceding claim, wherein the catheter is no more than 50 cm long.

23. An intermittent catheter assembly according to any preceding claim, wherein the catheter is formed of a polyolefin material, preferably polyethylene and/or polypropylene.

24. An intermittent catheter assembly according to any preceding claim, wherein the intermittent catheter is formed of a thermoplastic elastomeric material.

25. An intermittent catheter assembly according to any preceding claim, wherein at least 10% of the surface of the fluid collection bag is opaque.

26. An intermittent catheter assembly according to any preceding claim, wherein the fluid collection bag comprises a hand strap.

27. An intermittent catheter assembly according to claim 26, wherein the fluid collection bag receives fluid via an inlet arranged at an edge of the fluid collection bag, and the hand strap is arranged at the same edge as the inlet.

28. An intermittent catheter assembly according to claim 26 or 27, wherein the hand strap is arranged at an opposite end of the fluid collection bag to the outlet valve.

29. An intermittent catheter assembly according to any of claims 26 to 28, wherein the hand strap is arranged at an upper edge of the fluid collection bag and the outlet valve is arranged proximal to a base of the fluid collection bag.

30. An intermittent catheter assembly according to claims 2 and 26, wherein the hand strap is arranged toward the first lateral edge or toward the right lateral edge of the fluid collection bag, and the outlet valve is arranged off-centre toward the lateral edge of the fluid collection bag toward which the hand strap is arranged.

31. A packaged intermittent catheter assembly comprising the catheter assembly of any of claims 1 to 30 arranged within a pouch.

32. A packaged intermittent catheter assembly according to claim 31, wherein the pouch is sterile and sealed.

33. A method of manufacturing an intermittent catheter assembly comprising the steps of providing a catheter having a proximal end for insertion into the body and a distal end, and a fluid collection bag, arranging the fluid collection bag to receive fluid from the distal end of the catheter, wherein the fluid collection bag comprises an outlet valve configured to control release of fluid out of the fluid collection bag in use, the outlet valve comprising: a valve base attached to a panel of the fluid collection bag, the valve base comprising a base opening for receiving a liquid output from the fluid collection bag; and an outlet element configured to be laterally rotatable relative to the valve base from a closed configuration to an open configuration.

34. The method of claim 33, wherein the intermittent catheter assembly is the intermittent catheter assembly of any of claims 1 to 30.

35. A method of unpackaging a packaged intermittent catheter assembly, the packaged intermittent catheter assembly comprising an intermittent catheter assembly comprising the steps of providing a catheter having a proximal end for insertion into the body and a distal end, and a fluid collection bag, arranging the fluid collection bag to receive fluid from the distal end of the catheter, wherein the fluid collection bag comprises an outlet valve configured to control release of fluid out of the fluid collection bag in use, the outlet valve comprising: a valve base attached to a panel of the fluid collection bag, the valve base comprising a base opening for receiving a liquid output from the fluid collection bag; and an outlet element configured to be laterally rotatable relative to the valve base from a closed configuration to an open configuration, wherein the catheter and the fluid collection bag are arranged within a pouch.

36. The method of claim 35, wherein the packaged intermittent catheter assembly is the packaged intermittent catheter assembly of claim 31 or 32.
